(19) United States
(12) Patent Application Publication
(10) Pub. No.: US 2013/0102680 A1
(43) Pub. Date: Apr. 25, 2013

(54) METHODS FOR SELECTIVE SILENCING OF SENSORY NEURONS

(71) Applicant: CORNELL UNIVERSITY, Ithaca, NY (US)

(72) Inventor: Huai-hu Chuang, San Francisco, CA (US)

(73) Assignee: CORNELL UNIVERSITY, Ithaca, NY (US)

(21) Appl. No.: 13/647,678

(22) Filed: Oct. 9, 2012

Related U.S. Application Data
(60) Provisional application No. 61/544,782, filed on Oct. 7, 2011.

(51) Int. Cl. C07C 233/38

(52) U.S. Cl. CPC C07C 233/38
USPC 514/627; 435/375; 435/29

(57) ABSTRACT

The present invention relates to a method of treating hyperalgesia in a subject, which involves administering to a subject an amount of a TRPV1 agonist compound that selectively inhibits hyperactive nociceptive neurons. The present invention also relates to a method of selectively inhibiting hyperactive nociceptive neurons. Also disclosed is a method for reducing the electrical activity of hypersensitive neurons sensitized to TRPV1. The present invention also relates to a method for identifying candidate compounds that selectively inhibit hyperactive nociceptive neurons. Also disclosed is a pharmaceutical composition.

This application claims the benefit of U.S. Provisional Patent Application Ser. No. 61/544,782, filed Oct. 7, 2011, which is hereby incorporated by reference in its entirety.

### FIELD OF THE INVENTION

The present invention relates to a method of treating hyperalgesia, a method of selectively inhibiting hyperactive nociceptive neurons, a method of reducing the electrical activity of hypersensitive neurons with sensitized TRPV1, and a method of identifying candidate compounds that selectively inhibit hyperactive nociceptive neurons.

### BACKGROUND OF THE INVENTION

The capsaicin receptor TRPV 1 is the principal transduction channel for nociception. Capsaicin, a small lipophilic molecule from hot chili peppers, acts on sensory neurons by opening TRPV1 channel. TRPV1 is activated by noxious temperatures (>43° C.) and serves as an integrator for major pain-producing signals (Caterina et al., “The Capsaicin Receptor: A Heat-Activated Ion Channel in the Pain Pathway,” Nature 389:816-824 (1997) and Tominaga et al., “The Cloned Capsaicin Receptor Integrates Multiple Pain-Producing Stimuli,” Neuron 21:531-543 (1998)). Inflammatory hyperalgesia is dramatically reduced in mice lacking TRPV1 (Caterina et al., “Impaired Nociception and Pain Sensation in Mice Lacking the Capsaicin Receptor,” Science 288:306-313 (2000) and Davis et al., “Vanilloid Receptor-1 is Essential for Inflammatory Thermal Hyperalgesia,” Nature 405:183-187 (2000)). Besides being an attractive target for pain management, TRPV1 regulates autonomic function, such as body temperature, blood vessel tone, and release of transmitters from sensory nerves (Szallasi et al., “Vanilloid (Capsaicin) Receptors and Mechanisms,” Pharmacol. Rev. 51:159-212 (1999); Gamse et al., “Substance P Release from Spinal Cord Slices by Capsaicin,” Life Sci. 25:629-636 (1979); Zygmunt et al., “Vanilloid Receptors on Sensory Nerves Mediate the Vasodilator Action of Anandamide,” Nature 400:452-457 (1999); Jancso-Gabor et al., “Stimulation and Desensitization of the Hypothalamic Heat-Sensitive Structures by Capsaicin in Rats,” J. Physiol. 208:449-459 (1970); and Gavva et al., “The Vanilloid Receptor TRPV1 is Tonically Activated In Vivo and Involved in Body Temperature Regulation,” J. Neurosci. 27:3366-3374 (2007)). TRPV1 activated by capsaicin at a concentration far below the pain-producing threshold triggers neuropeptide release (Zygmunt et al., “Vanilloid Receptors on Sensory Nerves Mediate the Vasodilator Action of Anandamide,” Nature 400:452-457 (1999)) and production of other second messengers (Yang et al., “Activation of TRPV1 by Dietary Capsaicin Improves Endothelium-Dependent Vasorelaxation and Prevents Hypertension,” Cell Metab. 12:130-141 (2010)). In light of the complexity of TRPV1 actions in physiology, one major challenge in developing TRPV1-based pain treatment is to target therapeutic compounds to selectively inhibit hyperactive nociceptive neurons while sparing nerves of normal thresholds for pain detection.

Capsaicin is among the most powerful chemical agonists of TRPV 1. Being small and hydrophobic, capsaicin crosses the plasma membrane readily to reach its intracellular ligand-binding site on TRPV1 (Jordt et al., “Molecular Basis for Species-Specific Sensitivity to ‘Hot’ Chili Peppers,” Cell 108:421-430 (2002) and Jung et al., “Capsaicin Binds to the Intracellular Domain of the Capsaicin-Activated Ion Channel,” J. Neurosci. 19:529-538 (1999)), leading to channel activation and cation permeation. TRPV1 activation rapidly depolarizes nerves to evoke acute pain sensation and allows Ca²⁺ entry to initiate downstream signaling events such as neuropeptide release or production of other second messengers. Nevertheless, TRPV1 activation facilitates cellular transport of organic cations such as tetra-ethylammonium (“TEA”), N-methyl-glucamine (“NMG”) (Hellwig et al., “TRPV1Acts as Proton Channel to Induce Acidification in Nociceptive Neurons,” J. Biol. Chem. 279:34553-34561 (2004)) and the quaternary ammonium Na+ channel blocker QX-314 (Binshtok et al , “Inhibition of Nociceptors by TRPV 1-Mediated Entry of Impermeant Sodium Channel Blockers,” Nature 449:607-610 (2007)), or even larger fluorescent organic dyes such as FM1-43 or YO-PRO-1 (Meyers et al., “Lighting Up the Senses: FM1-43 Loading of Sensory Cells Through Nonselective Ion Channels,” J. Neurosci. 23:4054-4065 (2003) and Chung et al., “TRPV1 Shows Dynamic Ionic Selectivity During Agonist Stimulation,” Nat. Neurosci. 11:555-564 (2008)). Therefore, transmembrane cation transport in TRPV1-expressing cells may provide an effective route for agonist-dependent delivery of charged therapeutic molecules. The transport property coupled to TRPV1 activation allows application of capsaicin to load membrane-impermeable QX-314 into primary sensory afferents to suppress thermal nociception (Binshtok et al , “Inhibition of Nociceptors by TRPV1-Mediated Entry of Impermeant Sodium Channel Blockers,” Nature 449:607-610 (2007)).

Since excessive TRPV1 activation causes pathological pain, ideal pain management requires selective inhibition of hyperactive pain-sensing neurons, but sparing normal nociception.

The present invention is directed to overcoming deficiencies in the art.

### SUMMARY OF THE INVENTION

One aspect of the present invention relates to a method of treating hyperalgesia in a subject. This method involves administering to a subject an amount of a TRPV1 agonist compound, where the TRPV1 agonist compound selectively inhibits hyperactive nociceptive neurons in the subject under conditions effective to treat hyperalgesia in the subject.

Another aspect of the present invention relates to a method of selectively inhibiting hyperactive nociceptive neurons. This method involves administering to cells an amount of a TRPV 1 agonist compound capable of selectively inhibiting hyperactive nociceptive neurons but not normal nociceptive neurons.

A further aspect of the present invention relates to a method for reducing the electrical activity of hypersensitive neurons with sensitized TRPV1. This method involves selectively contacting hyperactive nociceptive neurons with a compound that induces (i) a permeant block of the TRPV1 receptor pore and (ii) cellular Ca²⁺ influx and transport of large cationic molecules, thereby reducing the electrical activity of the hyperactive nociceptive neurons.

Another aspect of the present invention relates to a method for identifying candidate compounds that selectively inhibit hyperactive nociceptive neurons. This method involves providing a cell that expresses a TRPV1 receptor; contacting the cell with a candidate compound; and analyzing the ability of the candidate compound to induce cellular influx of Ca²⁺ into the cell and transport of large cationic molecules. A candidate compound capable of inducing cellular influx of Ca²⁺ into the cell and transport of large cationic molecules is identified as a compound that selectively inhibits hyperactive nociceptive neurons.

A further aspect of the present invention relates to a pharmaceutical composition comprising a compound having a formula:

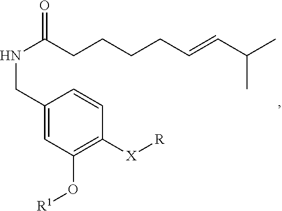

where
X is C(R¹)₂, O, N(R¹), or S;
R is —(CH₂)ₙYₐ(CH₂)ₘN(R¹)₃ or —C(O)(CH₂)ₙYₐ(CH₂)ₘN(R¹)₃;
R¹ is H or C₁₋₆ alkyl;
R² is H or C₁₋₆ alkyl;
R and R¹ are optionally substituted 1-6 times with halogen, ═O, —OR², —N(R²)₂, NO₂, C(O)R², C₂₋₆ alkenyl, and C₂₋₆ alkynyl;
n is an integer from 1 to 5;
m is an integer from 1 to 5;
a is 0 or 1; and
Y is O, N, or S,
   a stereoisomer, pharmaceutically acceptable salt, oxide, solvate, or ester thereof and a pharmaceutically acceptable carrier.

The present invention relates to activity-dependent TRPV1 agonists to identify nerves with excessive TRPV1 activity, as well as delivering charged anesthetics for neuronal silencing through the TRPV1 pore. Selective silencing of hyperactive nociceptive neurons in pain management requires use-dependent modulators that preferentially affect aberrantly sensitized TRPV1. To develop such use-dependent TRPV1 modulators, chemical derivatives of capsaicin (capsaicinoids) were synthesized and tested for whether they exhibited activity dependence. Several permanently charged capsaicinoids were found to activate TRPV1 via extracellular application, despite their predicted inability to cross the plasma membrane. Charged capsaicinoids retain substantial ability to induce Ca²⁺ influx and to transport large cationic molecules, but evoke rather small electric currents. Notably, activation of TRPV1 in intact cells by such cationic capsaicinoids obligates their own entry via TRPV1 pores. This pharmacokinetic property endows these membrane impermeable, water-soluble capsaicin derivatives the potential to act as activity-dependent drugs, since these cationic capsaicinoids will preferentially enter cells with elevated TRPV 1 activity to perpetuate further increase of membrane permeability to other therapeutic cations. Augmented permeation of large organic cations via TRPV1 sensitized by chemical messengers or other signaling pathways during inflammation or neuropathic pain may enable targeted delivery of cationic anesthetic compounds to selectively tame neurons with hyperactive TRPV1.

As described in the Examples, capsaicinoids of the present invention have the ability to induce YO-PRO-1 transport required permeation of both the agonist and the dye through the TRPV1 pore and could be enhanced by kinase activation or oxidative covalent modification. Moreover, cap-ET reduced capsaicin-induced currents by a voltage-dependent block of the pore. A low dose of cap-ET elicited entry of permanently charged Na+ channel blockers to effectively suppress Na+ currents in sensory neurons pre-sensitized with oxidative chemicals. The results shown herein implicate therapeutic potential of novel TRPV1 agonists exhibiting activity-dependent ion transport but with minimal pain-producing risks.

### BRIEF DESCRIPTION OF THE DRAWINGS

FIGS. 1A-D show that charged capsaicin derivates stimulate TRPV1-dependent calcium influx but fail to induce electrical currents. FIG. 1A shows the structures of three tetraakylammonium capsaicin derivatives bearing permanent positive charges. FIG. 1B is a graph showing representative traces of normalized Ca²⁺ signals induced by extracelluar application of capsaicin or derived analogues. Signals were normalized to the values from final application of a saturating dose of capsaicin. FIG. 1C shows dose response curves of evoked Ca²⁺ signals of three capsaicin analogues overlaid for comparison with the parental compound capsaicin. FIG. 1D is a graph showing that 100 μM cap-ET applied directly to the cytoplamsic side of an inside-out patch from a HEK cell expressing rat TRPV1 induced a barely detectable electric current compared to full activation of TRPV1 by 30 μM capsaicin. The TRPV1 current sensitized by 10 μM PAO, an oxidative chemical that potentiates the receptor, exhibited a recordable but small current activated by 100 μM cap-ET.

FIGS. 2A-E show that capsaicin opens the TRPV1 pore to facilitate cellular entry of YO-PRO-1. FIG. 2A is a pair of photographs showing that 1 μM capsaicin induced YO-PRO-1 uptake in rat TRPV1-expressing cells. FIG. 2B is a graph showing fluorescent signals quantified with plate-reader assays. TRPV 1 dependent fluorescent signals, indicating YO-PRO-1 entry, were concentration dependent. FIG. 2C is a chart showing application of the TRPV1 pore blocker ruthenium red (RR, 10 μM) suppressed capsaicin-mediated YO-PRO-1 uptake (upper, gray trace). To test the time dependence of ruthenium red block, cells were pre-incubated in capsaicin for two hours then the agonist was removed before the subsequent YO-PRO-1 uptake experiments. Ruthenium red (10 μM), when applied concomitantly with YO-PRO-1, quickly and effectively blocked the uptake (comparing the two black traces in the chart). FIG. 2D is a pair of graphs showing that pre-treatment with 200 μM Ba²⁺ also blocked capsaicin-induced YO-PRO-1 uptake (72.1±0.7% block n=3 wells). Ba²⁺ effects could be completely reversed (94.7±3.4% recovery, n=3 wells) by co-applying 1 mM EGTA, a chelator for this divalent cation, with YO-PRO-1. FIG. 2E shows that 5 μM YO-PRO-1 reversibly blocked TRPV1 currents evoked by 10 μM capsaicin in an inside-out patch. The i-V plot shows a weak voltage dependence of YO-PRO-1 block.

FIGS. 3A-B show that charged capsaicinoids evoke YO-PRO-1 uptake in TRPV1 expressing cells. In FIG. 3A, 20 μM extracellular cap-ET induced YO-PRO-1 uptake in 30 minutes. In FIG. 3B, agonist-induced fluorescence at the end of each experiment was normalized to total fluorescence from fixed cells in each well for derivation of dose response curves of each agonist. Relative efficacy of each agonist was displayed by assuming capsaicin as a full agonist.

FIGS. 4A-B show that block of the TRPV1 permeation pathway inhibits activation by cap-ET but not capsaicin. The upper panel illustrates that capsaicin can enter the cell by crossing membrane lipid bilayers, while cap-ET obligatorily uses the TRPV1 pore as the entry pathway. In FIG. 4A, TRPV1-expressing cells were incubated in 50 μM capsaicin or cap-ET for two hours. Then the agonist was removed before measuring kinetics of YO-PRO-1 entry. In FIG. 4B, co-incubation of 200 μM Ba²⁺ with 50 μM cap-ET for two hours blocked cap-ET induced YO-PRO-1 uptake nearly completely (black traces in the left chart). Even incubating the pre-treated cells with EGTA could not reverse the block.

FIG. 5 shows that cap-ET blocks TRPV1 pore in a voltage-dependent manner. Traces from capsaicin-induced currents in the absence (grey) or the presence (black) of cap-ET were displayed to demonstrate cap-ET block of capsaicin-induced TRPV1 currents. 100 μM cap-ET, when co-applied with 30 μM capsaicin, exhibited a strong voltage-dependent block from either outside (left) or inside (right). Cap-ET block was incomplete in either case. I-V curves were generated by normalizing the current amplitude at each voltage to the maximal TRPV1 current activated by 30 μM capsaicin at +100 mV.

FIGS. 6A-C show that PKC activation and receptor oxidation enhanced cap-ET evoked cation entry. FIGS. 6A-B show that treatment of TRPV1-expressing cells with 1 μM PDBu or 10 μM PAO increased the potency of capsaicin or cap-ET for YO-PRO-1 uptake. In FIG. 6C, cap-ET evoked substantial QX-314 entry in PAO sensitized neurons (filled circles) to reduce the current density of voltage gated Na+ channels compared with control groups (labeled with open circles) that showed no suppression (numbers of recording indicated in the parentheses, P<0.001, Mann-Whitney test). Representative traces of Na+ currents at −70, −10, and +20 mV from the medians of the un-inhibited and inhibited groups were shown on the top of the panel.

FIG. 7 is a graph showing the raw data of ratiometric Ca²⁺ images without normalization.

FIG. 8 is a graph showing capsaicin and capsaicinoid-evoked Ca²⁺ signals mediated by entry. Cells were initially incubated in a nominally Ca²⁺ free solution with agonists of specified concentrations for five minutes. At the end of 5-minute incubation, extracellular Ca²⁺ concentration was raised to 1 mM and robust signals were observed. No latency was observed when agonists had been pre-incubated.

FIG. 9 shows blow-up traces of concatenated traces evoked by repetitive voltage ramps displayed at high magnification. Direct application of cap-ET to the cytoplasmic face of the excised patches elicited currents without latency.

FIG. 10 shows that the rTRPV1 Y511A mutant channel can facilitate YO-PRO-1 entry, provided that the non-vanilloid ligand 2-aminoethoxydiphenyl borate (2-APB) was used to activate the receptor. For the wild type receptor, 2-APB and capsaicin elicited comparable YO-PRO-1 fluorescence. The bar graph shows the statistics of these experiments.

FIG. 11 is a graph showing that the kinetics of cap-ET-evoked YO-PRO-1 uptake is slower than that of capsaicin.

FIGS. 12A-B are graphs showing summarized statistics for PKC activation and receptor oxidation enhancement of cap-ET evoked YO-PRO-1 entry.

FIG. 13 is a table showing the water/octanol partition coefficient that was calculated as the ratio of the two measurements. The ratio (capET/capsaicin)=331±68.

### DETAILED DESCRIPTION OF THE INVENTION

A first aspect of the present invention relates to a method of treating hyperalgesia in a subject. Hyperalgesia is an increased sensitivity to pain or enhanced intensity of pain sensation, which may be caused by tissue damage-related activation of nociceptors or peripheral nerves.

A nociceptor (which is also referred to herein as a “nociceptive neuron”) is a sensory receptor that responds to potentially damaging stimuli by sending nerve signals to the spinal cord and brain. This process, called nociception, usually causes the perception of pain. In mammals, nociceptors are sensory neurons that are found in any area of the body that can sense pain either externally or internally. External examples are in tissues such as skin (cutaneous nociceptors), cornea, and mucosa. Internal nociceptors are in a variety of organs, such as the muscle, joint, bladder, gut, and continuing along the digestive tract.

Nociceptive neurons, respond to painful mechanical or thermal stimulation and chemical irritants. Thermal nociceptors are activated by noxious heat or cold at various temperatures. Mechanical nociceptors respond to excess pressure or mechanical deformation. Chemical nociceptors are TRP channels that respond to a wide variety of chemicals (e.g., capsaicin).

The method of treating hyperalgesia in a subject of the present invention involves administering to a subject an amount of a TRPV1 agonist compound, where the TRPV1 agonist compound selectively inhibits hyperactive nociceptive neurons in the subject under conditions effective to treat hyperalgesia in the subject.

TRPV1 is a nonselective cation channel that may be activated by a wide variety of exogenous and endogenous physical and chemical stimuli. The best-known activators of TRPV1 are heat greater than 43° C. and capsaicin. The activation of TRPV1 leads to a painful, burning sensation. Its endogenous activators include: low pH (acidic conditions), the endocannabinoid anandamide, and N-arachidonoyl-dopamine. TRPV 1 receptors are found mainly in the nociceptive neurons of the peripheral nervous system, but they have also been described in many other tissues, including the central nervous system. TRPV 1 is involved in the transmission and modulation of pain (nociception), as well as the integration of diverse painful stimuli.

The sensitivity of TRPV1 to noxious stimuli is not static. Upon tissue damage and the consequent inflammation, a number of inflammatory mediators, such as various prostaglandins and bradykinin, are released. These agents increase the sensitivity of nociceptors to noxious stimuli. This manifests as an increased sensitivity to painful stimuli (hyperalgesia) or pain sensation in response to non-painful stimuli allodynia. Most sensitizing pro-inflammatory agents activate the phospholipase C pathway. Phosphorylation of TRPV1 by protein kinase C has been shown to play a role in sensitization of TRPV1, the cleavage of PIP2 by PLC-beta can result in disinhibition of TRPV 1 and consequently contribute to the sensitivity of TRPV1 to noxious stimuli.

Agonists such as capsaicin and resiniferatoxin activate TRPV 1 and, upon prolonged application, TRPV 1 activity decreases (desensitization), leading to alleviation of pain. Agonists have been applied locally to the painful area through a patch or an ointment. Numerous capsaicin-containing creams are available over-the-counter, containing low concentrations of capsaicin (0.025-0.075%). Novel preparations containing higher capsaicin concentration (up to 10%) are under clinical trials. 8% capsaicin patches have recently become available for clinical use, with supporting evidence demonstrating that a 30 minute treatment can provide up to 3 months analgesia by causing regression of TRPV1 containing neurons in the skin.

In contrast to TRPV 1 agonists currently being used to treat hyperalgesia, TRPV1 agonist compounds of the present invention selectively inhibit hyperactive nociceptive neurons to treat hyperalgesia. By “selectively inhibit” it is meant that the TRPV1 agonist compounds of the present invention target nociceptive neurons with excessive (i.e., at a level that is above normal TRPV 1 nociceptive activity) TRPV 1 activity and deliver charged anesthetics for neuronal silencing, while sparing normal nociception.

TRPV1 agonist compounds of the present invention, which are used to selectively inhibit hyperactive nociceptive neurons, have the ability to exploit the TRPV 1 pore to deliver the agonist itself and other charged anesthetics for neuronal silencing.

In one embodiment, the TRPV1 agonist compound of the present invention induces Ca²⁺ influx into a nociceptive neuron. In another embodiment, the TRPV1 agonist compound induces entry into hyperactive nociceptive neurons via the TRPV1 pore of permanently charged Na+ channel blockers to suppress Na+ currents in sensory neurons. In yet another embodiment, the TRPV1 agonist compound enters hyperactive nociceptive neurons through an activated TRPV1 pore. In a further embodiment, the TRPV1 agonist compound may be a permanent blocker of the TRPV1 pore in hyperactive nociceptive neurons. In another embodiment, the TRPV1 agonist compound is hydrophilic. In a further embodiment, the TRPV1 agonist compound is impermeable to a plasma membrane of neurons.

The TRPV1 agonist compound of the present invention may, according to one embodiment, be a permanently charged capsaicin derivative of the formula:

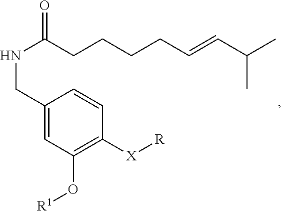

where
X is C(R¹)₂, O, N(R¹), or S;
R is —(CH₂)ₙYₐ(CH₂)ₘN(R¹)₃ or —C(O)(CH₂)ₙYₐ(CH₂)ₘN(R¹)₃;
R¹ is H or C₁₋₆ alkyl;
R² is H or C₁₋₆ alkyl;
R and R¹ are optionally substituted 1-6 times with halogen, ═O, —OR², —N(R²)₂, NO₂, C(O)R², C₂₋₆ alkenyl, and C₂₋₆ alkynyl;
n is an integer from 1 to 5;
m is an integer from 1 to 5;
a is 0 or 1; and
Y is O, N, or S
   a stereoisomer, pharmaceutically acceptable salt, oxide, solvate, or ester thereof.

Specific TRPV1 agonist compounds, which are permanently charged capsaicin derivatives, include, without limitation, the compounds cap-ET and cap-ETEA, having the following structures:

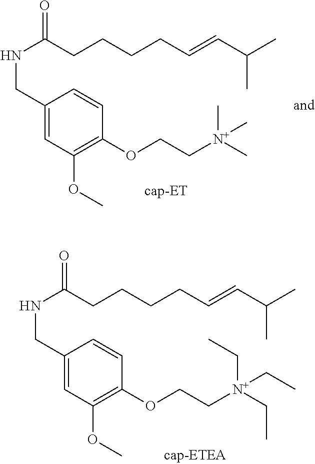 [SMILES: COc1cc(CNC(=O)CCCC/C=C/C(C)C)ccc1OCC[N+](C)(C)C ; CC[N+](CC)(CC)CCOc1ccc(CNC(=O)CCCC/C=C/C(C)C)cc1OC]

A further aspect of the present invention relates to a pharmaceutical composition comprising a compound of the formula:

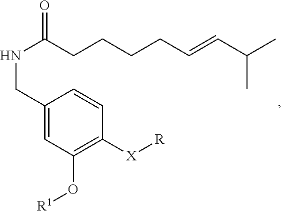

where
X is C(R¹)₂, O, N(R¹), or S;
R is —(CH₂)ₙYₐ(CH₂)ₘN(R¹)₃ or —C(O)(CH₂)ₙYₐ(CH₂)ₘN(R¹)₃;
R¹ is H or C₁₋₆ alkyl;
R² is H or C₁₋₆ alkyl;
R and R¹ are optionally substituted 1-6 times with halogen, ═O, —OR², —N(R²)₂, NO₂, C(O)R², C₂₋₆ alkenyl, and C₂₋₆ alkynyl;
n is an integer from 1 to 5;
m is an integer from 1 to 5;
a is 0 or 1; and
Y is O, N, or S
   a stereoisomer, pharmaceutically acceptable salt, oxide, solvate, or ester thereof and a pharmaceutically acceptable carrier.

As would be understood by a person of ordinary skill in the art, the recitation of a “compound” is intended to include salts, solvates, oxides, and inclusion complexes of that compound as well as any stereoisomeric form, or a mixture of any such forms of that compound in any ratio. Thus, in accordance with some embodiments of the invention, a compound as described herein, including in the contexts of pharmaceutical compositions and methods of treatment is provided as the salt form.

The term “solvate” refers to a compound in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed.

The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. The term “pharmaceutically acceptable carrier” is used to mean any carrier, diluent, adjuvant, excipient, or vehicle, as described herein, keeping in mind that compounds of the present invention have a charge that creates high water solubility. Thus, in formulating the compounds of the present invention, it should be kept in mind that the compound's charge makes it useful, so excessive derivation of the composition rendering its action independent of its high water solubility could cause a loss of the compound's “selective” silencing of abnormally hyperactive neurons. This should be avoided in formulating pharmaceutical compositions of the present invention.

Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “prodrug” means compounds that are rapidly transformed in vivo to yield the parent compound of the above formula, for example by hydrolysis in blood.

Functional groups which may be rapidly transformed, by metabolic cleavage, in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bio availability as a result of enhanced solubility and/or rate of absorption conferred upon the parent compound by virtue of the presence of the metabolically cleavable group. A thorough discussion of prodrugs is provided in the following: “Design of Prodrugs,” H. Bundgaard, ed., Elsevier (1985); “Methods in Enzymology,” K. Widder et al, Ed., Academic Press, 42, p.309-396 (1985); “A Textbook of Drug Design and Development,” Krogsgaard-Larsen and H. Bundgaard, ed., Chapter 5; “Design and Applications of Prodrugs,” p.113-191 (1991); “Advanced Drug Delivery Reviews,” H. Bundgard, 8, p.1-38 (1992); Journal of Pharmaceutical Sciences, 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds of the invention. In designing prodrugs for use with the present invention, cleavage should occur outside the cell.

TRPV1 agonist compounds of the present invention (i.e., that selectively inhibit hyperactive nociceptive neurons) can be formulated into a composition that comprises a carrier, preferably a pharmaceutically acceptable carrier. Such compositions can be in solid or liquid form, such as tablets, capsules, powders, solutions, suspensions, or emulsions.

The TRPV1 agonist compounds and/or pharmaceutical compositions containing the TRPV1 agonist compounds may be orally administered, for example, with an inert diluent or with an assimilable edible carrier, may be enclosed in hard or soft shell capsules, may be compressed into tablets, or may be incorporated directly with food. For oral therapeutic administration, the TRPV1 agonist compounds of the present invention may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to an active ingredient, sucrose as a sweetening agent, methyl and propylparabens as preservatives, a dye, and flavoring such as cherry or orange flavor.

TRPV1 agonist compounds of the present invention may also be administered parenterally. Solutions or suspensions of the compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The TRPV1 agonist compounds of the present invention may also be administered directly to the airways in the form of an aerosol or other inhalable formulation. For use as aerosols, the agent of the present invention in solution or suspension may be packaged in a pressurized aerosol container together with suitable propellants, for example, hydrocarbon propellants like propane, butane, or isobutane with conventional adjuvants. The TRPV1 agonist compound of the present invention also may be administered in a non-pressurized form such as in a nebulizer or atomizer. An inhalable formulation typically is in the form of an inhalable powder, which may include a pharmaceutically acceptable carrier. Suitable pharmaceutically acceptable carriers for inhalable powders may be composed of any pharmacologically inert material or combination of materials which is acceptable for inhalation. Advantageously, the carrier particles are composed of one or more crystalline sugars; the carrier particles may be composed of one or more sugar alcohols or polyols. In one embodiment, the carrier particles are particles of dextrose or lactose. Conventional dry powder inhalers include the Rotohaler, Diskhaler, and Turbohaler. The particle size of the carrier particles may range from about 10 microns to about 1000 microns. Alternatively, the particle size of the carrier particles may range from about 20 microns to about 120 microns. In certain embodiments, the size of at least 90% by weight of the carrier particles is less than 1000 microns and preferably lies between 60 microns and 1000 microns. The relatively large size of these carrier particles gives good flow and entrainment characteristics. Where present, the amount of carrier particles will generally be up to 95%, for example, up to 90%, or up to 80% or up to 50% by weight based on the total weight of the powder. The amount of any fine excipient material, if present, may be up to 50% or up to 30%, or up to 20%, by weight, based on the total weight of the powder.

Sustained release formulations include implantable devices that include a slow-dissolving polymeric matrix and one or more TRPV1 agonist compounds retained within the polymeric matrix. The matrix can be designed to deliver substantially the entire payload of the vehicle over a predetermined period of time, such as about one to two weeks up to about one to three months.

Although the formulations and compositions can also be delivered topically, it is also contemplated that the compositions can be delivered by various transdermal drug delivery systems, such as transdermal patches as known in the art.

In addition, the TRPV1 agonist compounds of the present invention can be administered using a delivery vehicle for passive or targeted delivery to particular cells that are known to possess hyperactive nociceptive neurons. Any suitable passive or targeted delivery vehicle can be employed, including liposomes, polymeric nanoparticles, polyethylene glycol conjugates, and cell uptake peptides.

Targeting the delivery vehicle to a cell of interest is typically achieved through the use of antibodies, binding fragments thereof, or nucleic acid aptamers that are bound or suspended to the surface of the delivery vehicle.

Polymeric nanoparticles can be targeted to cell-surface markers using aptamers designed using the SELEX procedure (Farokhzad et al., “Targeted Nanoparticle-aptamer Bioconjugates for Cancer Chemotherapy In Vivo,” Proc. Natl. Acad. Sci. USA 103(16):6315-6320 (2006), which is hereby incorporated by reference in its entirety). Nanoparticles and microparticles may comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath et al., “Biodegradable Polymeric Nanoparticles as Drug Delivery Devices,” J. Control Release 70(1-2):1-20 (2001), which is hereby incorporated by reference in its entirety. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen et al., “Colloids and Surfaces,” Biointerfaces 16(1-4):3-27 (1999), which is hereby incorporated by reference in its entirety. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(D,L-lactide) may be used while the lipid monolayer is typically composed of a mixture of lipids. Suitable methods of preparation are similar to those referenced above for polymer nanoparticles. Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese and Morpurgo, “Bioconjugation in Pharmaceutical Chemistry,” IL Farmaco 54(8):497-516 (1999), which is hereby incorporated by reference in its entirety.

The appropriate dose regimen, the amount of each dose administered, and specific intervals between doses of the active compound will depend upon the particular active compound employed, the conditions of the patient being treated, and the nature and severity of the disorder or conditions being treated. Preferably, the active compound is administered in an amount and at an interval that results in the desired treatment of or improvement in the disorder or condition being treated (e.g., hyperalgesia).

As one skilled in the art will readily appreciate, the compounds of the present invention can be used alone or in combination with other treatments as a combination therapy.

As used herein, “treating” or “treatment” may involve stopping or reversing progression of a condition or disorder, or controlling symptoms thereof. As it pertains to hyperalgesia, treating includes reducing sensitivity to pain or lessening pain intensity.

In carrying out this method of the present invention, a subject to be treated may include, without limitation, any mammal, including, for example, a cow, horse, pig, sheep, goat, dog, cat, rabbit, rodent, non-human primate, or, in a preferred embodiment, a human.

Another aspect of the present invention relates to a method of selectively inhibiting hyperactive nociceptive neurons. This method involves administering to cells an amount of a TRPV 1 agonist compound capable of selectively inhibiting hyperactive nociceptive neurons but not normal nociceptive neurons.

In one embodiment, this method of the present invention is carried out in vitro, such as in a sample. In vitro methods may be carried out to test the activity of certain compounds and/or pharmaceutical compositions against cells in, e.g., a solution or a tissue sample, for their ability to selectively inhibit hyperactive nociceptive neurons but not normal nociceptive neurons.

In another embodiment, this method of the present invention is carried out in vivo in an animal or patient or subject. In carrying out this method in vivo, the administering step is carried out, e.g., by administering an agent (i.e., a TRPV1 agonist compound) orally, intradermally, intramuscularly, intraperitoneally, intravenously, subcutaneously, or intranasally. The agent may be administered alone or with suitable pharmaceutical carriers, and can be in solid or liquid form, such as tablets, capsules, powders, solutions, suspensions, or emulsions.

A further aspect of the present invention relates to a method for reducing the electrical activity of hypersensitive neurons sensitized to TRPV1. This method involves selectively contacting hyperactive nociceptive neurons with a compound that induces (i) a permeant block of the TRPV1 receptor pore and (ii) cellular Ca²⁺ influx and transport of large cationic molecules, thereby reducing the electrical activity of the hyperactive nociceptive neurons.

Another aspect of the present invention relates to a method for identifying candidate compounds that selectively inhibit hyperactive nociceptive neurons. This method involves providing a cell that expresses a TRPV1 receptor; contacting the cell with a candidate compound; and analyzing the ability of the candidate compound to induce cellular influx of Ca²⁺ into the cell and transport of large cationic molecules. A candidate compound capable of inducing cellular influx of Ca²⁺ into the cell and transport of large cationic molecules is identified as a compound that selectively inhibits hyperactive nociceptive neurons.

Initial screening steps may involve testing the water solubility of compounds. This can be carried out, for example, by an octanol/water partitioning test where water and octanol are combined and a test compound is added to the solution. Phase separation by centrifugation can then be carried out, after which the two phases are analyzed for the concentration of test compound. Compounds with a high concentration in the water phase identify the compounds as water soluble. Water soluble compounds are then tested for biological activity, as discussed below.

In accordance with this aspect of the present invention, providing a cell that expresses a TRPV1 receptor can be carried out by using methods known and used by persons of ordinary skill in the art. In one embodiment, a nucleic acid molecule encoding the TRPV1 receptor can be introduced into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase.

These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK± or KS± (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cod Springs Harbor, N.Y. (1989), which is hereby incorporated by reference in its entirety.

A variety of host-vector systems may be utilized to express the protein-encoding sequence in a cell. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, e.g. AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others including, but not limited to, lacUV 5, ompF, bla, 1pp, and the like, may be used to direct high levels of transcription of adjacent DNA segments. Additionally, a hybrid trp-lacUV 5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno sequence about 7-9 bases 5′ to the initiation codon (e.g., ATG) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The protein-encoding nucleic acid, a promoter molecule of choice, a suitable 3′ regulatory region and, if desired, a reporter gene are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a protein (e.g., TRPV1 receptor) is inserted into a vector in the sense (i.e., 5′→3′) direction, such that the open reading frame is properly oriented for the expression of the protein under the control of a promoter of choice. Single or multiple nucleic acids may be ligated into an appropriate vector in this way, under the control of a suitable promoter, to prepare a nucleic acid construct.

Once the isolated nucleic acid molecule encoding the protein or polypeptide has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

Contact between candidate compounds and a cell model (i.e., a cell containing an expression system to express TRPV 1 protein) can be carried out as desired, including, but not limited to, in culture in a suitable growth medium for the cell. Alternatively, mice, rats or other mammals are injected with compounds to be selected.

Methods of identifying candidate compounds that selectively inhibit hyperactive nociceptive neurons can also be carried out in a cell-free format. For example, it may be desirable to confirm activity of compounds through electrophysiology methods. Such methods may involve removing the cell membrane from cells and directly applying a candidate compound to an intracellular site. Such tests eliminate the need for the test compound to enter the cell via a pore (e.g., TRPV1), which can take time.

The assay methods of the present invention can be accomplished in any vessel suitable for containing the reactants. Examples of such vessels include, without limitation, microtiter plates, test tubes, and micro-centrifuge tubes.

In another embodiment, this method further involves designing de novo compounds based on said identifying. Designing may involve, for example, linking functional groups or small molecule fragments of the identified compounds to form de novo compounds.

### EXAMPLES

The following examples are provided to illustrate embodiments of the present invention but are by no means intended to limit its scope.

### Example 1

### Synthesis of Charged Capsaicinoid Derivate Compounds

Boc-aminoethyl bromide (262 mg, 1.17 mmol, prepared from ethanolamine) was dissolved in 3 mL of dry tetrahydrofuran (THF) and added to capsaicin (305 mg, 1.0 mmol), potassium tert-butoxide (112 mg, 1.0 mmol), and 18-crown-6 (264 mg, 1.0 mmol) dissolved in 7 mL of dry THF. The reaction was stirred overnight, concentrated, and partitioned between EtOAc and saturated NH₄Cl solution. The EtOAc was washed well with water, then dried and concentrated to 439 mg of white solid. Of this, 150 mg (0.33 mmol) was dissolved in 1 mL of CH₂Cl₂, 35 μL (0.35 mmol) of thiophenol, and 0.5 mL of trifluoroacetic acid (TFA). After 30 minutes, the solvent was evaporated and the residue purified by reverse phase HPLC on a semi-preperative C₁₈ column, eluting with a linear gradient of 20 to 70% CH₃CN in 50 mM NH₄OAc, pH 4.5, over 50 minutes. Cap-ethylamine (cap-EA) eluted at 50% CH₃CN and unreacted capsaicin at 68%. Fractions were lyophilized twice to a white powder (81 mg, 54%). ¹H NMR (DMSO-d₆, 400 MHz) showed additional peaks relative to capsaicin at δ4.66 (t, 2H) and 3.39 (t, 2H). MS calcd for C₂₀H₃₃N₂O₃ (MH)+:349.2. Found: 349.2.

The acetate salt of cap-ET (46 mg, 0.11 mmol) was further dissolved in 2 mL of dry dimethylformamide (DMF). Diisopropylethylamine (65 μL, 0.37 mmol) and iodomethane (22 μL, 0.35 mmol) were added and the reaction was stirred overnight. HPLC using the conditions described above gave a cap-ET peak eluting at 46% CH₃CN. Fractions were lyophilized twice to a white powder (39 mg, 91%). ¹H NMR (DMSO-d₆, 400 MHz) showed additional peaks relative to capsaicin at δ4.52 (t, 2H), 3.30 (t, 2H), and 3.22 (s, 9H). MS calcd for C₂₃H₃₉N₂O₃⁺ (M)+: 391.3. Found: 391.3.

Cap-BT was similarly prepared by full methylation of cap-butylamine, and cap-ETEA was prepared by full ethylation of cap-ethylamine using ethyl iodide.

### Example 2

### Water/Octanol Partition

A 10 μL aliquot of 10 mM cap-ET in water or 50 mM capsaicin in octanol was added to 0.5 mL of 10 mM NaH₂PO₄ (pH 7.4) and 0.5 mL of octanol, which formed two layers. The tubes were vortexed well and centrifuged briefly to separate the layers. UV absorbance (at 280 nm) of the top (octanol) and bottom (water) layers was measured in a spectrophotometer.

### Example 3

### Cell Culture

rTRPV1 Stable Line

Human embryonic kidney (HEK) 293 cells stably expressing rat TRPV1 were grown in DMEM/F12 with 10% newborn calf serum and antibiotics and cultured in a 5% CO₂ aerated incubator at 37° C. Cells were trypsinized and plated on 96-well plates or matrix-coated coverslips for Ca²⁺ imaging, YO-PRO-1 imaging, plate-reader assays, or electrophysiological recording. Calcium imaging, fluorescent dye uptake assays, and electrophysiological experiments were all performed 24-72 hours after plating.

### Culture of Primary Sensory Neurons

Newborn rats (postnatal day 0-2, Rattus norvegicus) were euthanized by acute decapitation following guidelines of Institutional Animal Care and Use Committee of Cornell University. Dorsal root ganglia were dissected out, rinsed with Hanks' buffer and digested in the same solution containing 1 mg/ml type 1 collagenease (Worthington Biochemical Corporation) for 15-30 minutes at 37° C. Partially digested tissues were triturated with plastic pipette tips to release neurons into the suspension. The debris was discarded. Neurons in the suspension were centrifuged at 1,000 rpm for 3-5 minutes. Pellets containing neurons and fibroblasts were re-suspended in 0.25% trypsin and digested for additional 5 minutes at 37° C. Cells were spun down, aliquotted onto coverslips coated with poly-D-lysine (0.1 mg/ml), and cultured in MEM supplemented with 10% newborn calf serum and Glutamine in an incubator (5% CO₂, 37° C.).

### Example 4

### Electrophysiology of HEK293 Cells

Inside-out or cell-attached membrane patches were formed in cells expressing rat TRPV1 bathed in a standard solution containing 10 mM HEPES (Na), 140 mM NaCl, 1 mM MgCl₂, and 0.1 mM EGTA, pH to 7.4. The extracellular (pipette) solution had a similar composition to the standard bath solution except 0.1 mM EGTA was replaced with 1 mM CaCl₂. For the experiments to determine extracellular block of 100 μM cap-ET, standard bath solution was used as the pipette solution. The solution for agonist perfusion further replaced all chloride ions with glucaonate. The patch pipettes were pulled from borosilicate capillaries and fire-polished until final tip diameters of 8-12 μm were achieved with access resistance of 0.2-0.3 MΩ. Data were filtered at 2 kHz and sampled at 1 kHz using the Pulse-Pulsefit (HEKA, GmBH) software. Patch membranes were held either at constant membrane potential (−60mV) or given a 320-ms ramp pulse from −120 to +80 mV every second for continuous recordings. For derivation of voltage-current (i-V) relationships, 200-msec voltage steps of 10 mV decrements (ranging from +100 mV to −110 mV) were applied to membrane patches. Amplitudes of steady-state currents were used to plot against potentials of test pulses for construction of all i-V plots.

### Example 5

### Neuronal Na⁺ Currents

Pretreatment for cap-ET and/or QX-314 Loading

Neurons were incubated in artificial cerebral spinal fluid (ACSF) with the following composition (in mM) 10 HEPES, 10 glucose, 150 NaCl, 4.4 KCl, 1.2 MgCl₂, and 1 Na₂HPO₄, pH 7.4. The agonist or blocker was loaded in two stages. In the first stage, neurons were incubated in ACSF containing either PAO (10 μM) or cap-ET (10 μM), or a combination of both (10 μM PAO+10 or 50 μM cap-ET) for 15 minutes. The coverslips were then rinsed with ACSF extensively. In the second phase, cells were incubated in 5 mM QX-314 (in ACSF) for another 15 minutes.

### Whole-cell Recording

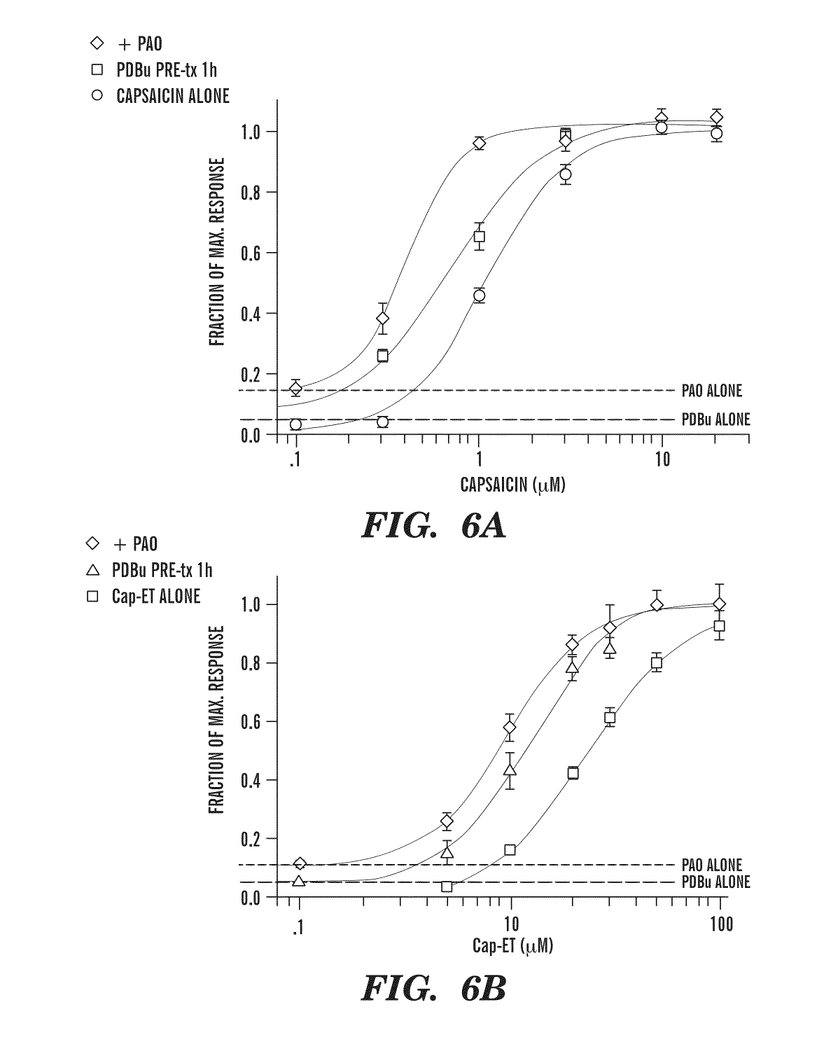

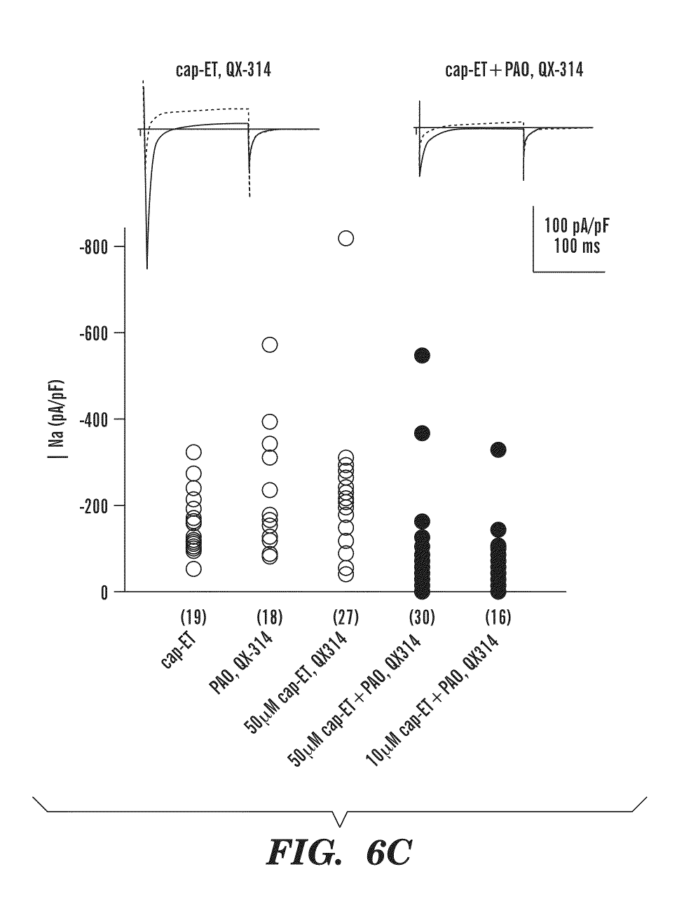

After finishing loading chemicals into neurons, the coverslips were rinsed in ACSF several times, and transferred to the recording chamber containing the following extracellular solution composed of (in mM) 10 HEPES, 10 glucose, 60 NaCl, 60 NMGCl, 4.4. KCl, 1 MgCl₂, 0.1 CdCl₂, 15 TEA, and 5 4-aminopyridine, pH 7.4. Neurons were recorded using the conventional tight seal whole cell voltage clamp method. The intracellular solution for recording contained (in mM) 110 CsCl, 2 MgCl₂, 1 CaCl₂, 11 EGTA, and 10 HEPES, pH adjusted to 7.4 with about 25 mM CsOH. Neuronal Na⁺ currents were recorded after breaking into the whole cell mode, immediately following capacitance cancellation, and using a protocol with 200-ms voltage steps from -70 to +30 mV. Patch pipette resistances were in the range of 2-3 MΩ. Maximal peak amplitudes of Na⁺ currents were measured and divided by cellular capacitance to obtain current density. All neuronal data were plotted on the chart in FIG. 6C. Each circle represented one neuron.

### Example 6

### Ca²⁺ Imaging

HEK293 cells were loaded with 5 μM Fura-2 AM and 0.01% pluronic acid in Ca²⁺ imaging buffer for 3 hours. The imaging solution contained 8.5 mM HEPES, 140 mM NaCl, 3.4 mM KCl, 1.7 mM MgCl₂, and 1 mM CaCl₂, pH to 7.4 with NaOH. 2× solution containing different concentrations of capsaicin and capsaicin analogues was pipetted into individual wells for agonist delivery. The images were acquired every 2 seconds for capsaicin and every 5 seconds for synthetic analogues. The plots displayed mean±s.e.m values of F340/F380 (150 ms exposure time for each wavelengths) ratios from fields containing 180 to 400 cells.

### Example 7

### YO-PRO-1 Imaging

Cells were plated in 96-well plates to near confluence. Images were acquired at a rate of 1 frame per 15 sec. The fields were illuminated for 2 sec for each frame. The fluorescent intensity was measured at the emission wavelength of 510/20 nm. 2× concentration of agonists and 10 μM YO-PRO-1 were mixed and added at equal volume to a bath solution with the same electrolyte composition except omitting CaCl₂.

### Example 8

### Plate-reader Assays

TRPV1 cell lines were passed into 96-well plate, grown to confluence, and assayed in a physiological Ringer solution. 50 μl of solution as used for YO-PRO-1 imaging with agonists of various concentrations was added to each well. Fluorescence intensity (485-nm excitation, 516-nm emission) was monitored by Multi-detection Microplate Reader (BioTek). For normalization, cells were fixed by 5% PFA at the end of experiments and counted total fluorescence from each well after adding digitonin solution (Assay Designs) with 5 μM YO-PRO-1.

### Example 9

### Extracellular Application of Charged Capsaicinoids Activated TRPV1

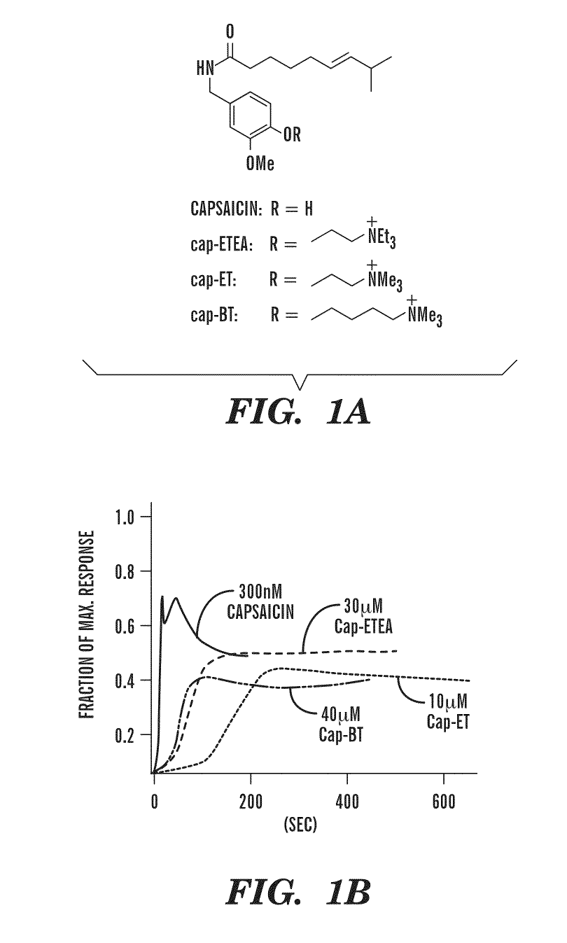

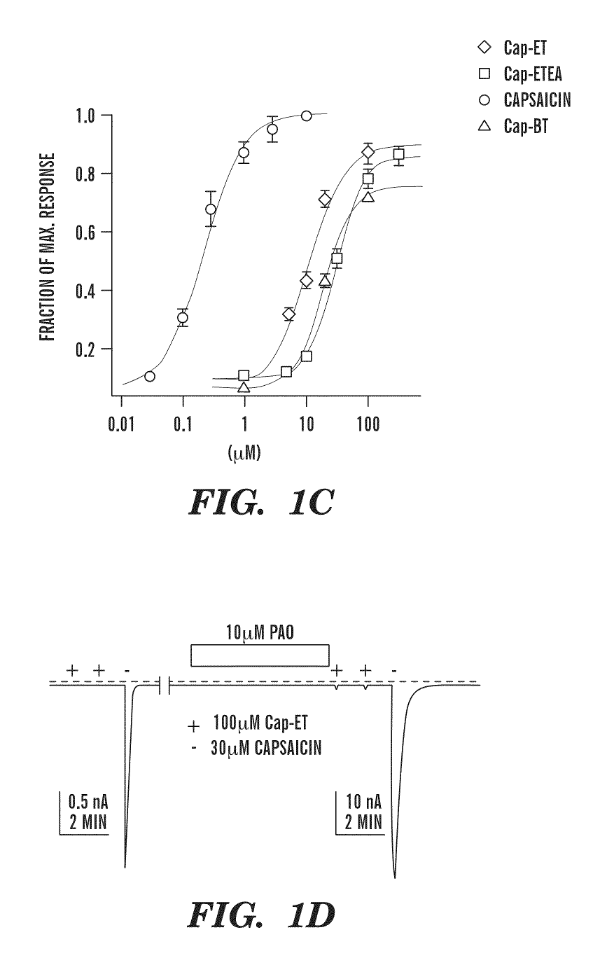

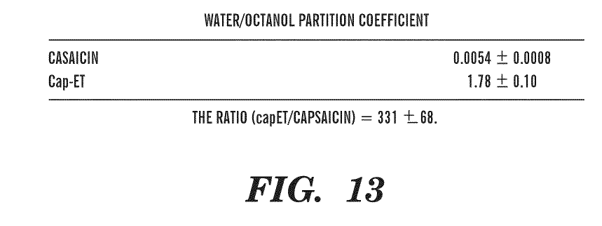

The capsaicinoids shown in FIG. 1 were prepared by alkylation of the potassium phenolate salts of capsaicin, as described above. Three hydrophilic quaternary ammonium capsaicin derivatives were synthesized: capsaicin O-ethyl (trimethyl-ammonium) acetate (cap-ET), capsaicin O-butyl(trimethylammonium) acetate (cap-BT), and capsaicin O-tetra-ethylammonium acetate (cap-ETEA) (FIG. 1A). All compounds were purified by reverse phase chromatography under conditions that ensured their complete separation from any remaining capsaicin, and all showed satisfactory proton NMR and mass spectral analysis. These three capsaicin derivatives are fully charged cationic quaternary ammonium salts, using the same functionality that renders the cysteine-reactive reagent MTSET membrane impermeable (Akabas et al., “Acetylcholine receptor Channel Structure Probed in Cysteine-Substitution Mutants,” Science 258:307-310 (1992) and Stauffer et al., “Electrostatic Potential of the Acetylcholine Binding Sites in the Nicotinic Receptor Probed by Reactions of Binding-Site Cysteines with Charged Methanethiosulfonates,” Biochem. 33:6840-6849 (1994)). Cap-ET and cap-BT differ in the distance of the trimethylammonium group from the vanilloid ring, while cap-ETEA is a more hydrophobic triethylammonium derivative. Based on water/octanol partitioning, cap-ET is over 300-fold more hydrophilic than capsaicin (FIG. 13).

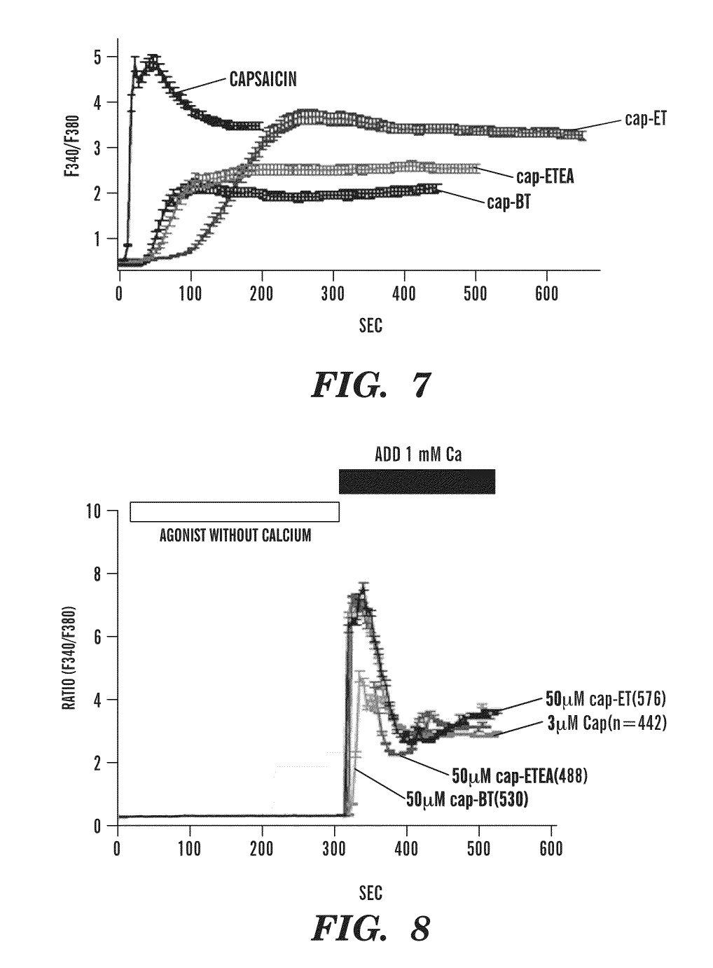

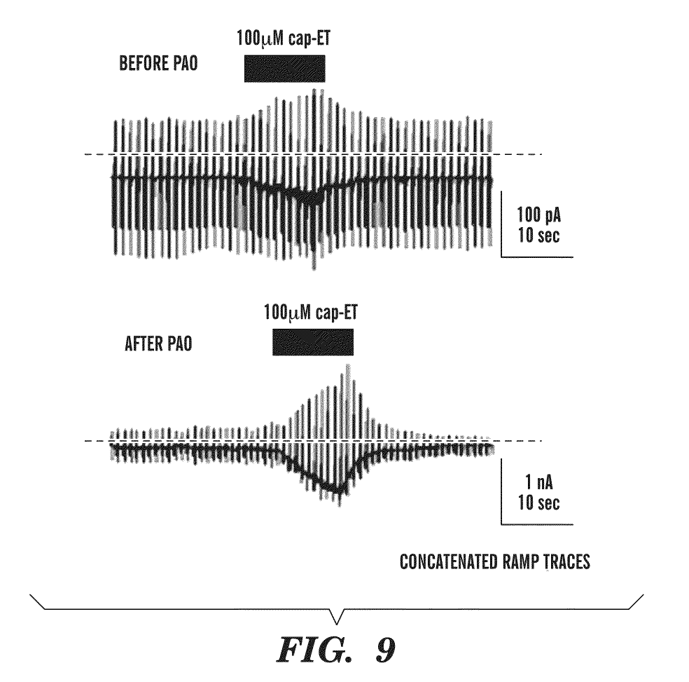

When applied extracellularly, capsaicin readily crosses the plasma membrane to reach its intracellular ligand binding site on TRPV 1 to activate the receptor to prompt a large intracellular Ca²⁺ rise, evidenced by a robust fluorescent signal in ratiometric fura-2 imaging (FIG. 1B). It was predicted that extracellular application of permanently charged capsaicinoids would fail to activate TRPV1, since the agonists would fail to access intracellular vanilloid binding sites. Surprisingly, extracellular application of cap-ET, cap-BT, and cap-ETEA still effectively raised intracellular Ca²⁺ levels (FIG. 1B). The activation is absolutely extracellular Ca²⁺ dependent, with cap-ET being the most potent and efficacious among the three (FIG. 1C, FIG. 7, and FIG. 8). Comparable to previous studies, alkylation of capsaicin at the phenolic oxygen reduces the potency of these compounds relative to capsaicin (Walpole et al., “Analogues of Capsaicin with Agonist Activity as Novel Analgesic Agents; Structure-Activity Studies. 1. The Aromatic ‘A-Region’,” J. Med. Chem. 36:2362-2372 (1993)): Cellular responses evoked by charged TRPV1 agonists exhibited much slower kinetics even when these agonists were applied at a concentration of several orders of magnitude higher than capsaicin. In contrast, direct application of cap-ET to excised inside-out membrane patches from HEK cells opened TRPV1 with no delay by electrophysiological measurements (FIG. 1D, FIG. 9). However, ionic currents evoked by 100 μM cap-ET were much smaller than those by capsaicin (Icap-ET/Icap=1.1 ±0.3% at −60 mV, n=11) even under recording conditions in which no receptor desensitization occurs. The low efficacy of cap-ET to induce currents persisted even for TRPV 1 maximally sensitized by phenylarsine oxide (PAO), a cysteine-reacting chemical mimicking cellular oxidative stress (Icap-ET/Icap=3.0±0.9% at −60 mV, n=6, after 5 minutes PAO sensitization, FIG. 1D) (Chuang et al., “Oxidative Challenges Sensitize the Capsaicin Receptor by Covalent Cysteine Modification,” Proc. Natl. Acad. Sci. U.S.A. 106:20097-20102 (2009)). Slow activation kinetics of cationic capsaicin analogs in Ca²⁺ imaging is, therefore, not a trivial outcome of reduced potency or efficacy of charged capsaicinoids, but more likely a consequence of delayed access of these agonists to their intracellular ligand binding sites. Even though the permanently charged capsaicinoids have dramatically reduced hydrophobicity, which is generally considered to be important for agonist efficacy of TRPV1 ligands (Walpole et al., “Analogues of Capsaicin with Agonist Activity as Novel Analgesic Agents; Structure-Activity Studies. 1. The Aromatic ‘A-Region’,” J. Med. Chem. 36:2362-2372 (1993); Walpole et al., “Analogues of Capsaicin with Agonist Activity as Novel Analgesic Agents; Structure-Activity Studies. 2. The Amide Bond ‘B-Region’,” J. Med. Chem. 36:2373-2380 (1993); and Walpole et al., “Analogues of Capsaicin with Agonist Activity as Novel Analgesic Agents; Structure-Activity Studies. 3. The Hydrophobic Side-Chain ‘C-Region’,” J. Med. Chem. 36:2381-2389 (1993)), their successful activation of TRPV1 predicts sufficient cellular bioavailability and, therefore, a potential therapeutic usefulness of these compounds.

The ability of permanently charged capsaicinoids to access the intracellular ligand-binding site via extracellular application raises the question of how these charged molecules cross biological membranes. Carrying the same positive tetraalkylammonium groups that severely impaired membrane permeability when attached to methanesulfonate (MTS) reagents, these synthetic cationic capsaicinoids cannot cross the lipid bilayer efficiently by passive diffusion. Alternative entry pathways were thus considered: active transport across the bilayer and permeation through the channel pore.

### Example 10

### Capsaicin-induced Cellular YO-PRO-1 Entry Is a Consequence of YO-PRO-1 Permeation Through the TRPV1 Pore

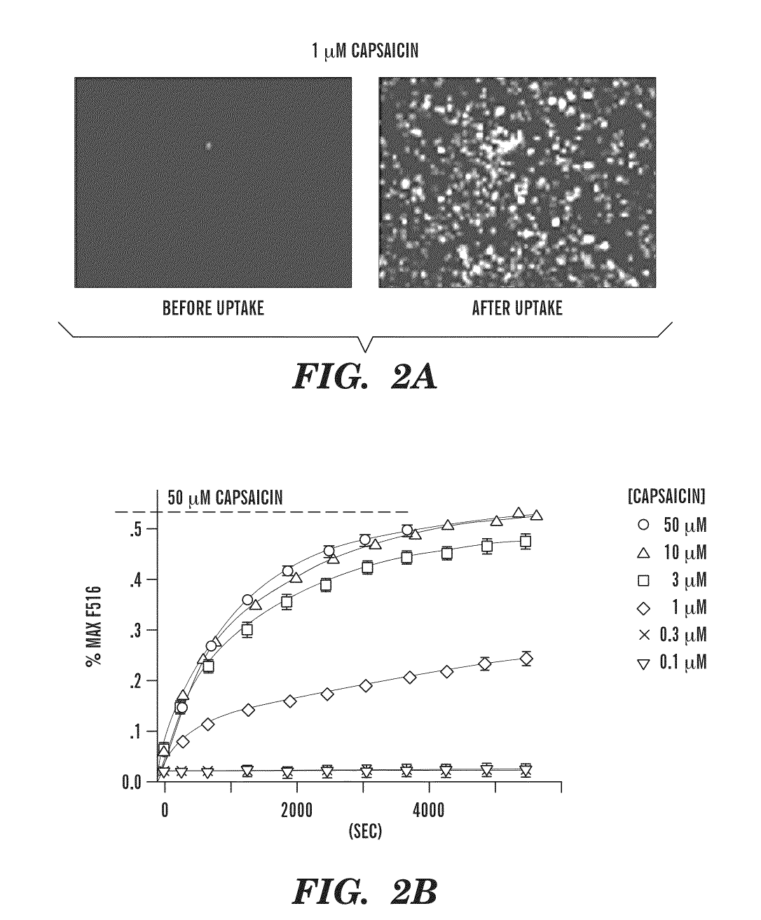

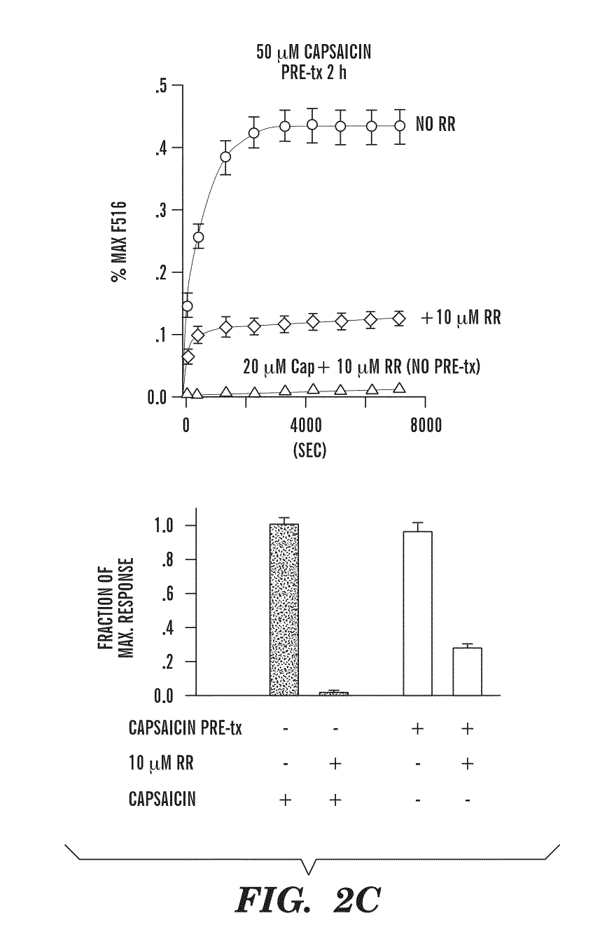

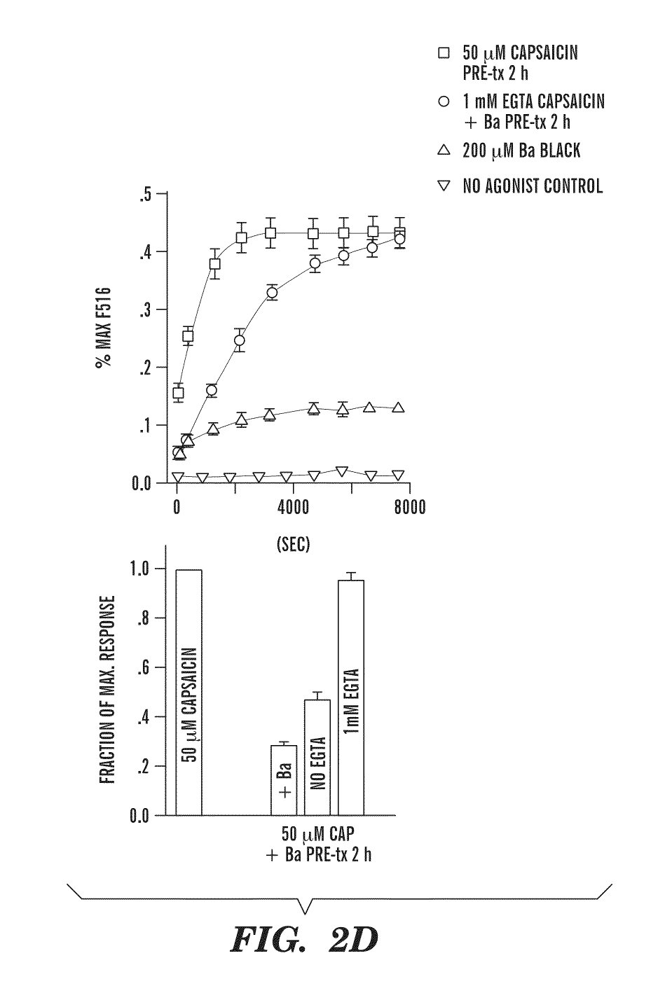

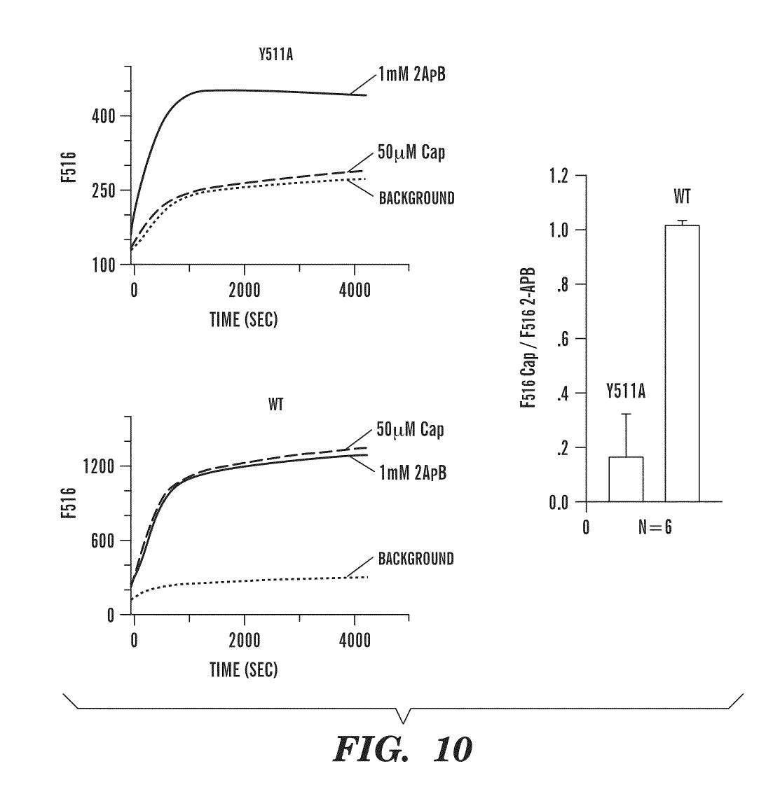

The TRPV1 pore is non-selective enough to allow the passage of structurally unrelated organic cations, including TEA and NMG (Hellwig et al., “TRPV1 Acts as Proton Channel to Induce Acidification in Nociceptive Neurons,” J. Biol. Chem. 279:34553-34561 (2004)). Capsaicin activation of TRPV1 was noted to elicit cellular entry of even larger cationic molecules, including the quaternary ammonium Na+ channel blocker QX-314 (Binshtok et al , “Inhibition of Nociceptors by TRPV1-Mediated Entry of Impermeant Sodium Channel Blockers,” Nature 449:607-610 (2007)), the biscationic styryl dye FM1-43 (Meyers et al., “Lighting Up the Senses: FM1-43 Loading of Sensory Cells Through Nonselective Ion Channels,” J. Neurosci. 23:4054-4065 (2003)), and the di-cyanine dye YO-PRO-1 (Chung et al., “TRPV1 Shows Dynamic Ionic Selectivity During Agonist Stimulation,” Nat. Neurosci. 11:555-564 (2008)). It is conceivable that charged capsaicinoids of comparable molecular weights may enter TRPV1-expressing cells via the same mechanism for transport of other organic cations. Thus, YO-PRO-1 was used as a reporter to elucidate the general mechanism of TRPV1-agonist dependent organic cation transport. YO-PRO-1 entered cytoplasm of TRPV1-expressing cells upon agonist treatment, while control cells without TRPV1 showed little YO-PRO-1 accumulation (FIGS. 2A-B and FIG. 10). Receptor activation is essential since capsaicin failed to elicit YO-PRO-1 transport in HEK cells expressing rTRPV1 Y511A (FIG. 10). Capsaicin-induced YO-PRO-1 fluorescence developed much slower compared with Ca²⁺ transients as expected from a larger molecular size of YO-PRO-1.

Therefore, tests were done to determine whether the TRP channel pore blocker ruthenium red (RR, 10 μM) inhibits YO-PRO-1 transport. It was found that RR effectively suppressed YO-PRO-1 entry (FIG. 2C). TRPV1-expressing cells were then pre-treated with 50 μM capsaicin for one hour, and it was found that they could efficiently take up YO-PRO-1 even without concurrent presence of extracellular capsaicin afterwards (FIG. 2C). In contrast, application of 10 μM RR following pretreatment with capsaicin for one hour caused an immediate and strong inhibition of YO-PRO-1 transport afterwards. An open TRPV 1 pore rather than an unrelated transporter activated downstream of the TRPV 1 signaling pathway appears to be the major determinant for the cellular transport of large organic cations. This predicts that cells pretreated with both 50 μM capsaicin and TRPV1 blockers should resume YO-PRO-1 transport as soon as the blocker is removed. Ruthenium red, however, has a relative high affinity to TRPV 1; RR cannot be effectively washed out to reverse the inhibition. Thus, Ba²⁺, a divalent ion that is more reversible but still effectively blocks YO-PRO-1 transport, was used to test whether the reversal of TRPV1 pore block restored permeation of large organic cations. Pre-incubation of cells in 50 μM capsaicin and 200 μM Ba²⁺ blocked subsequent YO-PRO-1 transport Inhibition of transport could be partially reversed by removing Ba²⁺ from the extracellular solution and almost completely recovered by incubating the cells pre-treated with capsaicin and Ba²⁺ in the chelator ethylene glycol tetraacetic acid (EGTA) (FIG. 2D). It was concluded that Ba²⁺ functions as a blocker within the TRPV1 pore to hinder YO-PRO-1 transport via the permeation pathway. Also, a reversible block of capsaicin-evoked TRPV1 currents by YO-PRO-1 was recorded, further suggesting a direct interaction of this dye with the pore (FIG. 2E). Agonist-dependent transport of large organic cations in TRPV1-expressing cells is mediated by their permeation through the channel pore, which could be the mechanism used by charged capsaicinoids for cellular entry.

### Example 11

### Cap-ET Crosses the TRPV1 Pore to Reach Cytoplasm for TRPV1 Activation

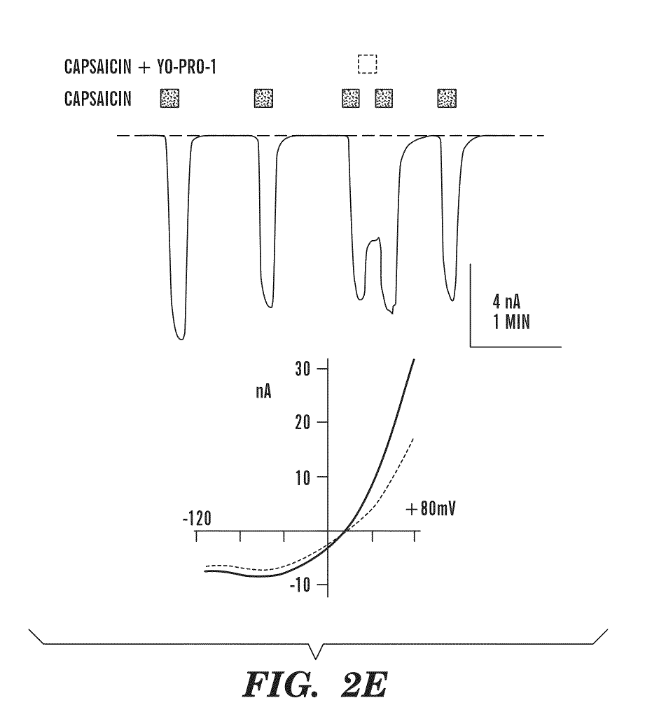

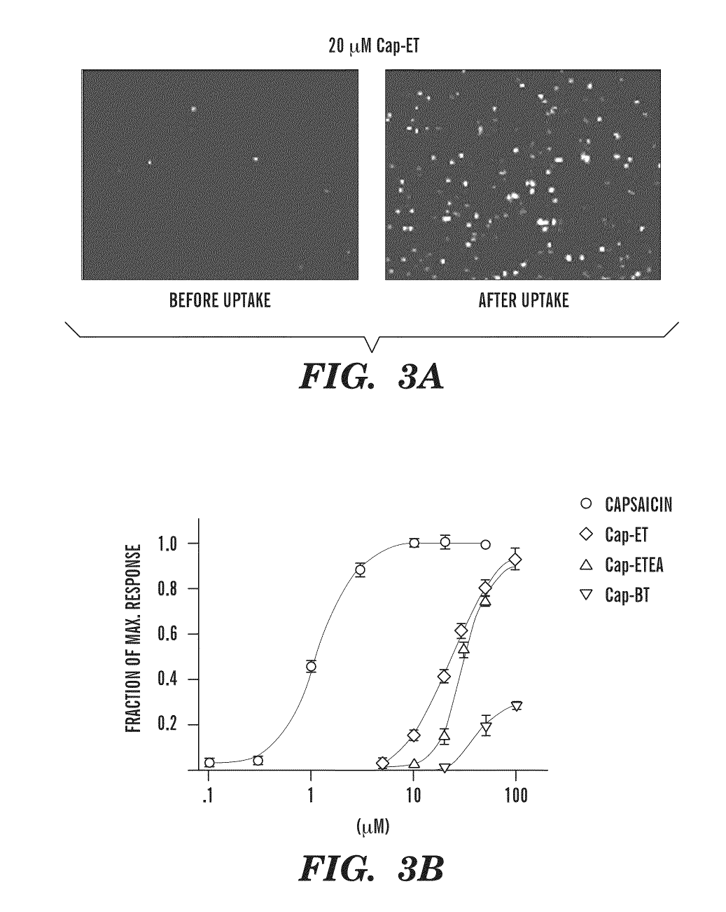

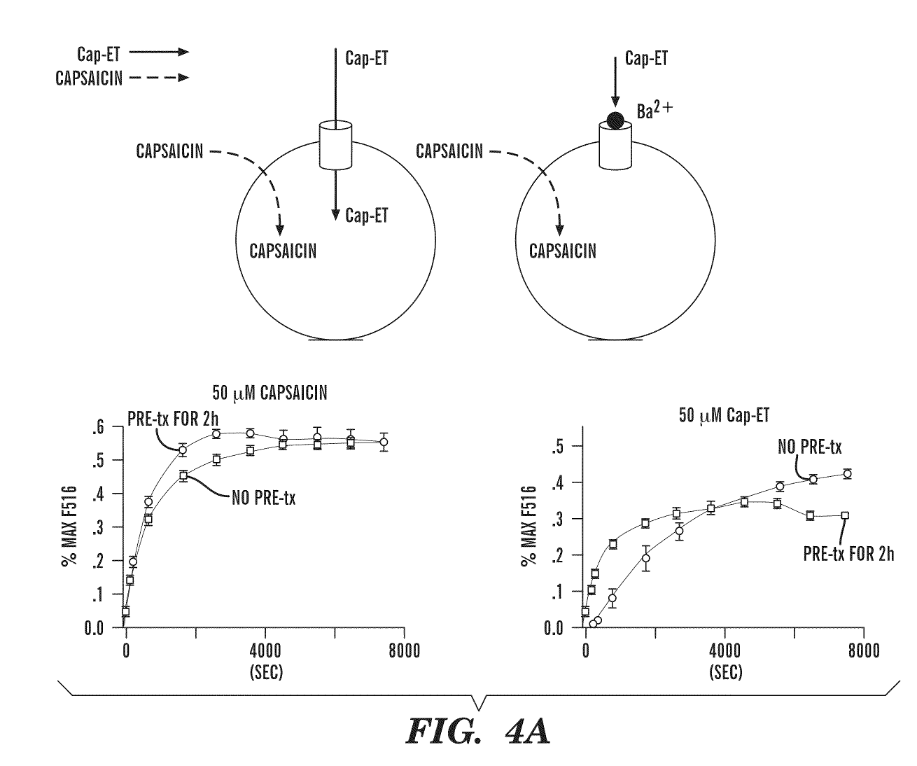

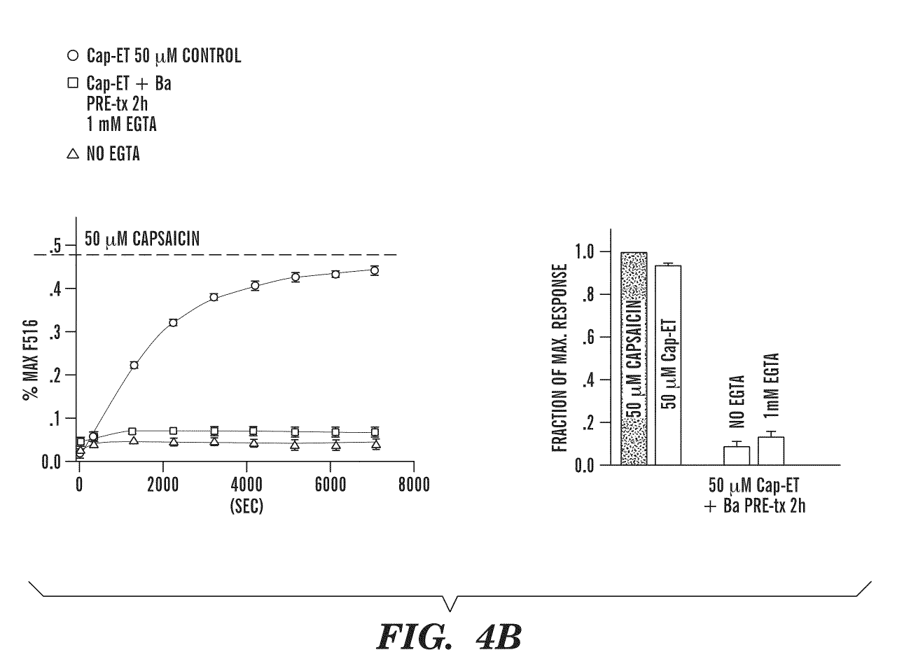

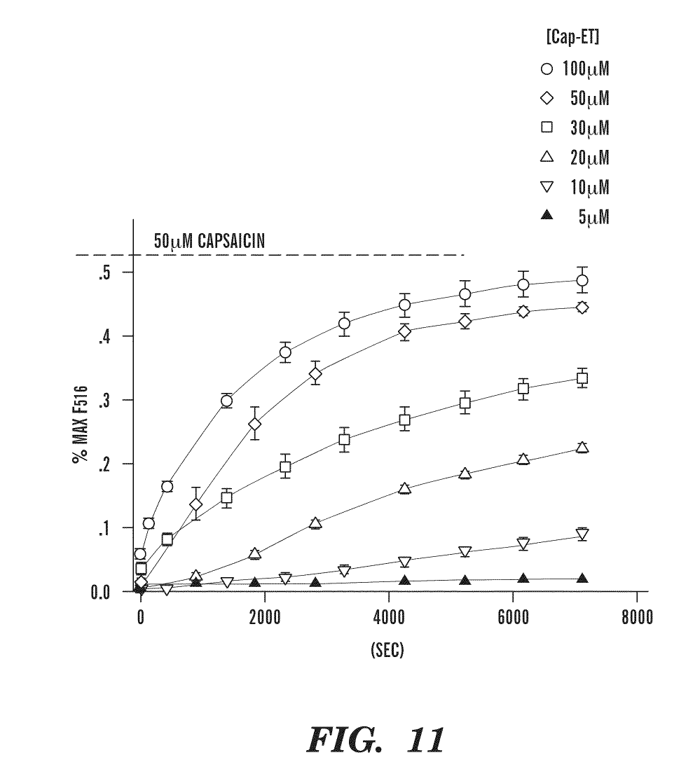

It was then asked whether extracellular application of permanently charged capsaicinoids could activate TRPV1 to facilitate YO-PRO-1 transport. All three charged capsaicinoids have considerable efficacies but show even slower kinetics compared to capsaicin (FIG. 3 and FIG. 11). Since cap-ET itself is a cation as large as YO-PRO-1, this charged capsaicinoid may enter cells by crossing the TRPV1 channel pore and then binding to its intracellular vanilloid-binding site to further increase channel opening. TRPV 1 expressing cells were pre-incubated in cap-ET for two hours to allow its accumulation inside cells. Cap-ET was then removed and 5 μM of YO-PRO-1 was added extracellularly. Pre-incubation of cap-ET enabled TRPV1-expressing cells to transport YO-PRO-1 as effectively and more swiftly: The kinetics of YO-PRO-1 fluorescence in cap-ET pretreated cells were much faster than cells without pre-treatment (5.37±0.99-fold decrease of t₁₁₂ to maximal fluorescence, compared to 1.14±0.04-fold in capsaicin treated wells, n=3 for each group, P=0.017, FIG. 4A), even though no extracellular cap-ET was given during the phase of YO-PRO-1 transport experiment. This result implies that cap-ET had been accumulated inside the cells to activate TRPV1 enough to increase the membrane permeability to YO-PRO-1, and consequently the accelerated entry. Given that Ba²⁺ can effectively inhibit the capsaicin-induced YO-PRO-1 entry, it was predicted that co-application of Ba²⁺ during the pre-incubation phase of cap-ET should effectively block permeation of this ligand and the consequent TRPV1 activation, thereby ablating YO-PRO-1 entry in the next step of experiments. Indeed, it was observed that cells pre-treated with cap-ET in the presence of 200 μM Ba²⁺ exhibited drastic reduction of YO-PRO-1 transport afterwards, whether 1 mM EGTA was provided during the transport experiments or not (FIG. 4B). Co-application of Ba²⁺ during pre-incubation of cap-ET hindered its own transport so much that an insufficient amount of agonist was accumulated inside cells. Taken together, these data indicate that charged capsaicinoids permeate the TRPV1 pore to access the intracellular capsaicin-binding site. Activity-dependent entry of TRPV 1 agonists via the channel pore might establish a positive feedback loop to sustain a long-lasting influx of YO-PRO-1 or other therapeutic organic cations, even for an agonist that activates electric currents poorly.

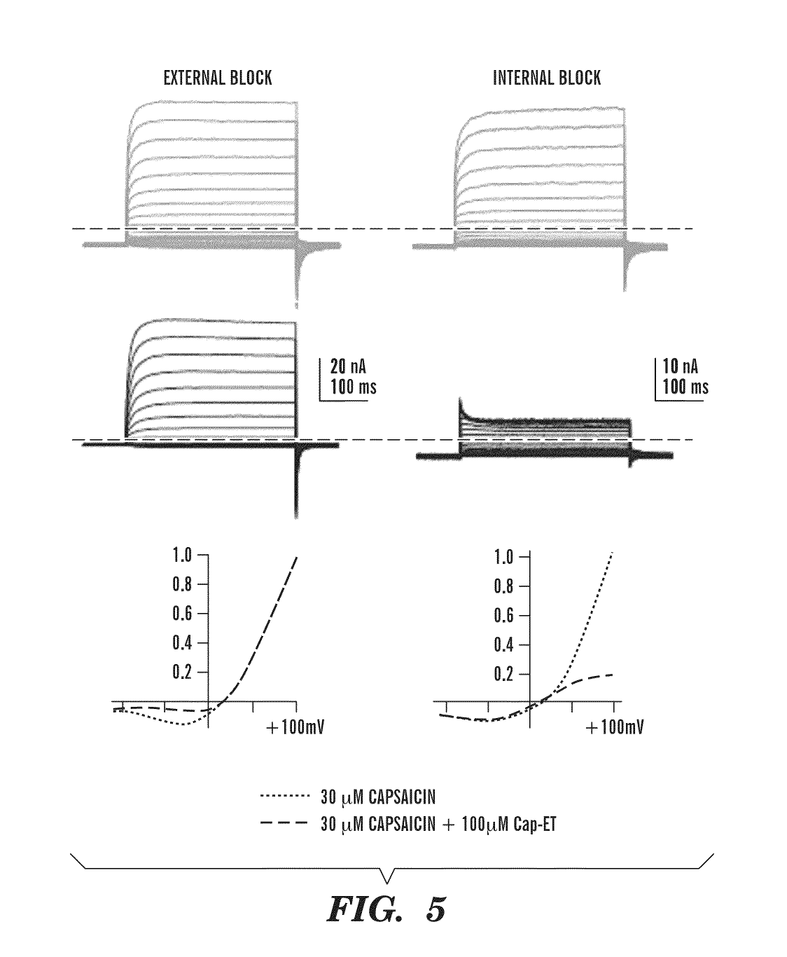

Transport of cap-ET via the channel pore suggests that this charged capsaicinoid should also reduce TRPV1 current by a pore blocking mechanism, in addition to its gating effect. Direct application of cap-ET alone to the cytoplasmic surface of an excised membrane patch induced rather small TRPV1 currents that exhibited a voltage-dependent pore block. To more accurately measure the extent of pore block by cap-ET, capsaicin was used to activate the receptor (FIG. 5). A saturating concentration of capsaicin robustly activated TRPV1 currents even in the presence of cap-ET, supporting the notion that capsaicin does interact with higher affinity to the vanilloid-binding site than cap-ET. Co-applied cap-ET exerted a substantial pore block with strong voltage-dependence, whether cap-ET was administered to the extracellular or the intracellular side. Given that cytoplasmic cap-ET only suppressed outward capsaicin-evoked TRPV1 currents, the inability of internal cap-ET to effectively activate TRPV1 currents in the negative voltage range must result from its low intrinsic efficacy. The voltage-dependent pore block by extracellular cap-ET may further reduce any inward TRPV1 currents. The residual ionic currents at extreme membrane potentials suggested that cap-ET block does not completely occlude the pore. These observations are mostly consistent with the idea that cap-ET, albeit a TRPV1 blocker, can still permeate an open TRPV 1 pore, provided that there is sufficient driving force.

### Example 12

### TRPV1 Sensitization Augments cap-ET Induced Cation Transport

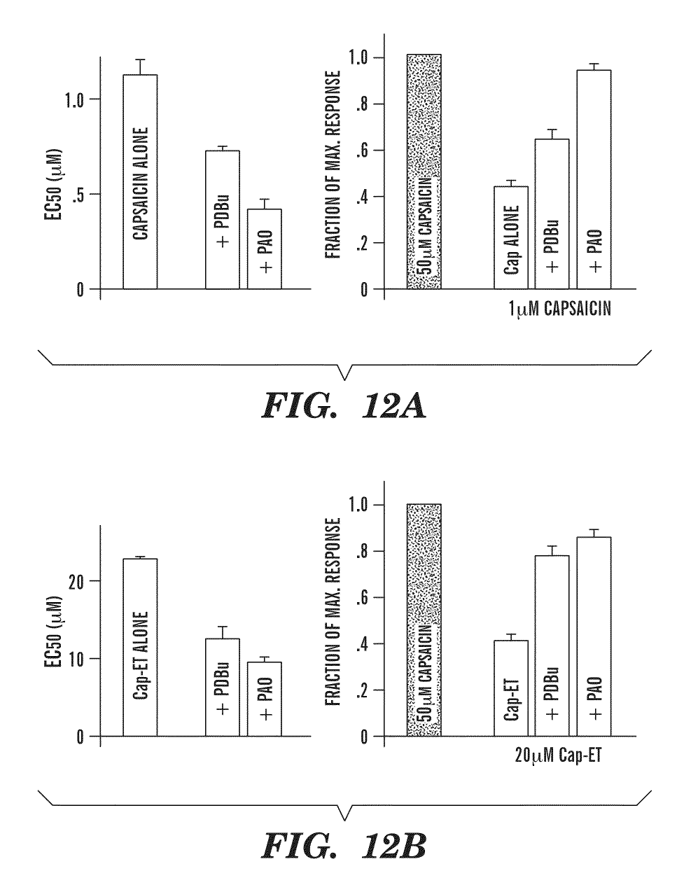

In many disease conditions, particularly the inflammatory states, TRPV1 becomes hypersensitized and triggers excessive pain. An agonist with the potential to stimulate activity-dependent therapeutic molecule transport will be most useful if it also exhibits a parallel increase of potency or efficacy for sensitized versus normal TRPV 1. Protein kinase C activation (Cesare et al., “A Novel Heat-Activated Current in Nociceptive Neurons and its Sensitization by Bradykinin,” Proc. Natl. Acad. Sci. U.S.A. 93:15435-15439 (1996); Cesare et al., “Specific Involvement of PKC-Epsilon in Sensitization of the Neuronal Response to Painful Heat,” Neuron 23:617-624 (1999); Premkumar et al., “Induction of Vanilloid Receptor Channel Activity by Protein Kinase C,” Nature 408:985-990 (2000); and Numazaki et al., “Direct Phosphorylation of Capsaicin Receptor VR1 by Protein Kinase Cepsilon and Identification of Two Target Serine Residues,” J. Biol. Chem. 277:13375-13378 (2002)) and oxidative modification (Chuang et al., “Oxidative Challenges Sensitize the Capsaicin Receptor by Covalent Cysteine Modification,” Proc. Natl. Acad. Sci. U.S.A. 106:20097-20102 (2009); Yoshida et al., “Nitric Oxide Activates TRP Channels by Cysteine S-Nitrosylation,” Nat. Chem. Biol. 2:596-60 (2006); and Salazar et al., “A Single N-Terminal Cysteine in TRPV1 Determines Activation by Pungent Compounds from Onion and Garlic,” Nat. Neurosci. 11:255-261 (2008)) are major biochemical pathways that enhance TRPV1 sensitivity to chemical agonists. PDBu and PAO, chemical activators stimulating PKC or mimicking cellular oxidation respectively, could elicit TRPV1-dependent intracellular Ca²⁺ rise (Chuang et al., “Oxidative Challenges Sensitize the Capsaicin Receptor by Covalent Cysteine Modification,” Proc. Natl. Acad. Sci. U.S.A. 106:20097-20102 (2009) and Bhave et al., “Protein Kinase C Phosphorylation Sensitizes but Does Not Activate the Capsaicin Receptor Transient Receptor Potential Vanilloid 1 (TRPV1),” Proc. Natl. Acad. Sci. U.S.A. 100:12480-12485 (2003)). They also dramatically shifted dose-response curves of agonist-evoked YO-PRO-1 entry leftward (FIGS. 6A-B and FIGS. 12A-B).

It was therefore tested whether cap-ET could present a prototype of TRPV1 agonists with therapeutic potential due to their preferential targeting of pathologically sensitized sensory nerves during inflammation or oxidative injuries. An effective reduction of voltage-gated Na+ currents was recorded by brief extracellular application of QX-314 in PAO-sensitized neurons pretreated with 10 or 50 μM cap-ET, while Na+ current densities from neurons treated with either PAO or cap-ET alone were comparable to cells not exposed to QX-314 (FIG. 6C). Taken together, TRPV1 sensitization could allow one to exploit this ion channel as the permeation pathway to deliver membrane impermeable local anesthetics preferentially into hyperactive nociceptors to suppress their electrical activity.

### Example 13

### Activity-Dependent Targeting of TRPV1 with a Pore-permeating Capsaicin Analog

The identification of TRPV1 has facilitated the development of novel analgesics (Szallasi et al., “The Vanilloid Receptor TRPV1: 10 Years from Channel Cloning to Antagonist Proof-of-Concept,” Nat. Rev. Drug Discov. 6:357-372 (2007) and Vay et al., “Current Perspectives on the Modulation of Thermo-TRP Channels: New Advances and Therapeutic Implications,” Exp. Rev. Clin. Pharmacol. 3:687-704 (2010)). However, most lead compounds developed for treating pain are TRPV1 antagonists that suppress channel activity without discriminating between normal and hyperactive TRPV 1. Alternatively, a permeation pathway for large organic ions coupled to ligand-gated channel activity, similar to that of ionotropic purinergic P2x receptors (Surprenant et al., “The Cytolytic P2Z Receptor for Extracellular ATP Identified as A P2X Receptor (P2X7),” Science 272:735-738 (1996); Virginio et al., “Pore Dilation of Neuronal P2X Receptor Channels,” Nat. Neurosci. 2:315-321 (1999); and Khakh et al., “Neuronal P2X Transmitter-Gated Cation Channels Change Their Ion Selectivity In Seconds,” Nat. Neurosci. 2:322-330 (1999)), represents an attractive conduit to introduce charged therapeutics into cytoplasms of select neuronal populations. For example, the mustard oil receptor TRPA1 responds to allyl-isothiocyanate to mediate YO-PRO-1 transport (Banke et al., “Dynamic Changes in the TRPA1 Selectivity Filter Lead to Progressive but Reversible Pore Dilation,” Am. J. Physiol. Cell Physiol. 298:C1457-68 (2010) and Chen et al., “Pore dilation Occurs in TRPA1 but Not In TRPM8 Channels,” Mol. Pain 5:3 (2009)). Moreover, anionic transmitters ATP or GABA can pass pores of pannexin or bestrophin respectively, underlying non-vesicular release of autocrine or paracrine factors in synapses (Schenk et al., “Purinergic Control of T Cell Activation by ATP Released Through Pannexin-1 Hemichannels,” Sci. Signal 1:ra6 (2008); Kronlage et al., “Autocrine Purinergic Receptor Signaling is Essential for Macrophage Chemotaxis,” Sci. Signal 3:ra55 (2010); Chekeni et al., “Pannexin 1 Channels Mediate ‘Find-Me’ Signal Release and Membrane Permeability During Apoptosis,” Nature 467:863-867 (2010); MacVicar et al., “Non-Junction Functions of Pannexin-1 Channels,” Trends Neurosci. 33:93-102 (2010); and Lee et al., “Channel-Mediated Tonic GABA Release from Glia,” Science 330:790-796 (2010)). Analogously, the remarkable permeability of the TRPV1 pore presents a novel route for delivery of membrane impermeable local anesthetics selectively into pain sensing neurons (Binshtok et al., “Inhibition of Nociceptors by TRPV1-Mediated Entry of Impermeant Sodium Channel Blockers,” Nature 449:607-610 (2007)).

Regardless of approach, pain alleviation strategies exploiting TRPV1 pharmacology still face two major issues: specificity and side effects. Both are related to the electrical excitation of sensory nerves downstream of TRPV 1 activation. Indiscriminate suppression of all TRPV 1 by receptor antagonists or TRPV 1 blockers cripples its protective role in alarming imminent or existing tissue damage. It is therefore advantageous to search for TRPV1 modulators that display activity-dependent efficacy. The strategy of co-administering capsaicin or strong TRPV1 agonists with membrane impermeable QX-314 to deliver local anesthetics into nerve terminals to reduce pain cannot bypass initial electric excitation induced pain before sufficient accumulation of this Na+ channel blocker to suppress action potentials; one cannot ease pain without eliciting it first. Besides high specificity and selectivity, an ideal TRPV 1 agonist used for stimulating pore-mediated transport of therapeutic organic cations should also retain the ability to activate chemical signals downstream of receptors, but cause little electrical excitation.

In the examples described herein, the possibility of designing TRPV1 drugs with these properties has been demonstrated. Simple chemical modification of the highly effective TRPV1 ligand capsaicin yields charged membrane-impermeable analogs. TRPV1 channels challenged with charged capsaicin derivatives exhibit a dramatic reduction of electrical current but preserve the ability to evoke Ca²⁺ transients and to transport large cationic molecules. Importantly, the efficacies of some charged analogs could approach that of capsaicin for Ca²⁺ signaling and cation transport. The special pharmacological property of these cationic capsaicin derivatives is a consequence of their permeation through TRPV1: The receptor ligand goes through the ion channel pore to directly access its own binding site. An activity-dependent permeation pathway can potentially permit sustained entry of externally applied charged capsaicinoids and Na+ channel blockers preferentially into hyperactive neurons without causing excessive electrical excitation.

Although an open TRPV1 pore is reported to be wide enough to accommodate large cations (Hellwig et al., “TRPV1 Acts as Proton Channel to Induce Acidification in Nociceptive Neurons,” J. Biol. Chem. 279:34553-34561 (2004); Meyers et al., “Lighting Up the Senses: FM1-43 Loading of Sensory Cells Through Nonselective Ion Channels,” J. Neurosci. 23:4054-4065 (2003); and Chung et al., “TRPV1 Shows Dynamic Ionic Selectivity During Agonist Stimulation,” Nat. Neurosci. 11:555-564 (2008)), the relative contribution of permeation through the TRPV1 pore as a cellular transport pathway of large cationic molecules has not been fully determined. The electrophysiological data described herein provides direct evidence that YO-PRO-1 and cap-ET do interact with the TRPV1 ion permeation pathway. Two pore blockers, RR and Ba²⁺, could also effectively suppress capsaicin-mediated YO-PRO-1 uptake. Both the rapid onset of RR block on capsaicin pre-treated cells and the reversal of Ba²⁺ inhibition by its removal or EGTA chelation suggest that the TRPV1 pore itself is the major entry pathway for YO-PRO-1 into the cell.

Cap-ET and related analogs have limited permeability across the lipid bilayer. Even if these ligands may enter cells via TRPV1 pores, one might question how these hydrophilic agonists could enter cells initially. Given that relative permeability of small metal ions for activated TRPV 1 is dependent on modes of channel activation (Tominaga et al., “The Cloned Capsaicin Receptor Integrates Multiple Pain-Producing Stimuli,” Neuron 21:531-543 (1998) and Samways et al., Tunable Calcium Current Through TRPV1 Receptor Channels,” J. Biol. Chem. 283:31274-31278 (2008)), chemical modulators might activate specific modes of TRPV1 opening and cause a rapid change of relative permeability of large organic cations (Chung et al., “TRPV1 Shows Dynamic Ionic Selectivity During Agonist Stimulation,” Nat. Neurosci. 11:555-564 (2008)). Such dynamic permeability change could not have happened before agonist application, that is, for hydrophilic cap-ET at starting time points of the YO-PRO-1 transport experiments. It is more likely that initial cap-ET entry was due to a sufficient basal channel activity of TRPV 1 at room temperature (Voets et al., “The Principle of Temperature-Dependent Gating in Cold- and Heat-Sensitive TRP Channels,” Nature 430:748-754 (2004); Ahern et al., “Voltage-Dependent Priming of Rat Vanilloid Receptor: Effects of Agonist and Protein Kinase C Activation,” J. Physiol. 545:441-451 (2002); and Matta et al., “Voltage is a Partial Activator of Rat Thermosensitive TRP Channels,” J. Physiol. 585:469-482 (2007)).

Although crystallographic data are not currently available to depict how organic cations permeate the TRPV 1 pore, large ions may pass the channel using a similar principle as adopted by the more selective K+ channels. That is, the entire permeation pathway may simultaneously accommodate multiple cations, which create local electrostatic interactions among different or the same species of cations (Doyle et al., “The Structure of the Potassium Channel: Molecular Basis of K+ Conduction and Selectivity,” Science 280:69-77 (1998) and Valiyaveetil et al., “Ion Selectivity in a Semisynthetic K+ Channel Locked in the Conductive Conformation,” Science 314:1004-1007 (2006)). Charged capsaicinoids possess aliphatic tails that might mediate hydrophobic interaction with the non-polar residues lining the pore also. Multiple ion bindings in the channel pore and hydrophobic interaction of aliphatic tails of charged capsaicinoids with other regions of the pore may collectively contribute to the permeation of cap-ET through TRPV1 channels. Further analysis will be required to delegate the contribution of each of these factors in the permeation of a charged capsaicinoid through TRPV1.

The charged capsaicinoids synthesized are all partial agonists. The relative reduction in efficacy, however, is assay-dependent. The remarkable permeability of TRPV1 to Ca²⁺ and receptor reserve may well be sufficient for a low efficacy agonist like cap-ET to elicit substantial Ca²⁺ rise to induce an effector response. The observation of a markedly reduced efficacy of cap-ET in electrophysiological studies compared to that in YO-PRO-1 transport experiments was, however, somewhat unanticipated. It is worth noting that the ability of TRPV 1 ligands to cause electrical excitation depends critically on the Na+ influx per second. In contrast, Ca²⁺ entry and YO-PRO-1 transport reflects the capacity of TRPV1 channels to serve as hydrophilic permeation pathways to allow gradual accumulation of cations inside cells over a long period of time. The relatively inefficient coupling to electrical currents suggests an intriguing possibility to apply a chemical agonist with a pharmacological profile similar to cap-ET to manage pain. In contrast with capsaicin that causes substantial initial irritation, cap-ET will have reduced risk to evoke pain. Despite the fact that cap-ET might still cumulate over a prolonged period to cause indiscriminate increase of membrane permeability even in non-sensitized nociceptors, the neuronal data suggested that cap-ET can distinguish sensitized versus non-sensitized neurons if applied within an appropriate therapeutic window. Charged capsaicinoids can also trigger Ca²⁺ dependent desensitization, which may also blunt TRPV1's ability to translate pain-producing stimuli into electric excitation. Moreover, extracellular cap-ET can block the inward current mediated by a strong pain-producing chemical like capsaicin. Unlike partial agonists generally exhibiting compromise in all downstream signaling pathways coupled to receptors, these charged synthetic ligands reveal a new avenue for agonist design. Their activity dependence for cellular entry and differential alteration of agonistic efficacy highlights the possibility to further engineer existing TRPV 1 ligands into ideal analgesics that can reduce pain arising from electrical activity without impairing signaling critical for other TRPV 1 functions.

What is claimed:

1. A method of treating hyperalgesia in a subject, said method comprising:
administering to a subject an amount of a TRPV1 agonist compound, wherein the TRPV1 agonist compound selectively inhibits hyperactive nociceptive neurons in the subject under conditions effective to treat hyperalgesia in the subject.

2. The method according to claim 1, wherein said TRPV1 agonist compound induces Ca²⁺ influx into a neural cell.

3. The method according to claim 1, wherein said TRPV1 agonist compound induces entry into hyperactive nociceptive neurons via the TRPVI pore of the TRPVI agonist itself and permanently charged Na⁺ channel blockers to suppress Na⁺ currents in sensory neurons.

4. The method according to claim 1, wherein said TRPV1 agonist compound enters hyperactive nociceptive neurons through an activated TRPV1 pore.

5. The method according to claim 1, wherein said TRPV1 agonist compound is a permeant blocker of the TRPV1 pore in hyperactive nociceptive neurons.

6. The method according to claim 1, wherein said TRPV1 agonist compound is impermeable to a plasma membrane of neurons.

7. The method according to claim 1, wherein said TRPV1 agonist compound is hydrophilic.

8. The method according to claim 1, wherein said TRPV1 agonist compound is a permanently charged capsaicin derivative.

9. The method according to claim 8, wherein said TRPV1 agonist compound is selected from cap-ET and cap-ETEA.

10. The method according to claim 1, wherein said administering is carried out orally, topically, transdermally, parenterally, subcutaneously, intravenously, intramuscularly, intraperitoneally, by intranasal instillation, by intracavitary or intavesical instillation, intraocularly, intraarterially, intralesionally, or by application to mucous membranes.

11. The method according to claim 1, wherein the subject is a mammal.

12. The method according to claim 1, wherein the subject is human.

13. The method according to claim 1, wherein the hyperalgesia is caused by thermal or chemical hypersensitivity.

14. A method of selectively inhibiting hyperactive nociceptive neurons, said method comprising:
administering to cells an amount of a TRPV 1 agonist compound capable of selectively inhibiting hyperactive nociceptive neurons but not normal nociceptive neurons.

15. The method according to claim 14, wherein said TRPV1 agonist compound induces Ca²⁺ influx into the hyperactive nociceptive neurons.

16. The method according to claim 14, wherein said TRPV1 agonist compound induces entry into the hyperactive nociceptive neurons via the TRPVI pore of the TRPV 1 agonist itself and permanently charged Na⁺ channel blockers to suppress Na⁺ currents in the hyperactive nociceptive neurons.

17. The method according to claim 14, wherein said TRPV1 agonist compound enters the hyperactive nociceptive neurons through an activated TRPV1 pore.

18. The method according to claim 14, wherein said TRPV1 agonist compound is a permeant blocker of the TRPV1 pore in the hyperactive nociceptive neurons.

19. The method according to claim 14, wherein said TRPV1 agonist compound is impermeable to the plasma membrane of neurons.

20. The method according to claim 14, wherein said TRPV1 agonist compound is hydrophilic.

21. The method according to claim 14, wherein said TRPV1 agonist compound is a permanently charged capsaicin derivative.

22. The method according to claim 14, wherein said TRPV1 agonist compound is selected from cap-ET and cap-ETEA.

23. The method according to claim 14, wherein said cells are in a subject and said administering is carried out orally, topically, transdermally, parenterally, subcutaneously, intravenously, intramuscularly, intraperitoneally, by intranasal instillation, by intracavitary or intavesical instillation, intraocularly, intraarterially, intralesionally, or by application to mucous membranes.

24. The method according to claim 23, wherein the subject is a mammal.

25. The method according to claim 23, wherein the subject is human.

26. A method for reducing the electrical activity of hypersensitive neurons with sensitized TRPV1, said method comprising:
selectively contacting hyperactive nociceptive neurons with a compound that induces (i) a permeant block of the TRPV1 receptor pore and (ii) cellular Ca²⁺ influx and transport of large cationic molecules, thereby reducing the electrical activity of the hyperactive nociceptive neurons.

27. The method according to claim 26, wherein the compound induces entry into the hyperactive nociceptive neurons via the TRPVI pore of permanently charged Na⁺ channel blockers to suppress Na⁺ currents in sensory neurons.

28. The method according to claim 26, wherein the compound enters the hyperactive nociceptive neurons through an activated TRPV1 pore.

29. The method according to claim 26, wherein the compound is a permeant blocker of the TRPV1 pore in the hyperactive nociceptive neurons.

30. The method according to claim 26, wherein the compound is impermeable to plasma membrane of neurons.

31. The method according to claim 26, wherein the compound is a permanently charged capsaicin derivative.

32. The method according to claim 26, wherein the compound is hydrophilic.

33. The method according to claim 26, wherein the compound is selected from cap-ET and cap-ETEA.

34. A method for identifying candidate compounds that selectively inhibit hyperactive nociceptive neurons, said method comprising:
providing a cell that expresses a TRPV1 receptor;
contacting the cell with a candidate compound; and
analyzing the ability of the candidate compound to induce cellular influx of Ca²⁺ into the cell and transport of large cationic molecules, wherein a candidate compound capable of inducing cellular influx of Ca²⁺ into the cell and transport of large cationic molecules is identified as a compound that selectively inhibits hyperactive nociceptive neurons.

35. The method according to claim 34, wherein the cell only expresses TRPV1 receptor.

36. The method according to claim 34 further comprising:
analyzing the ability of the candidate compound to induce a permeant block of a TRPV1 receptor pore.

37. The method according to claim 34, wherein the TRPV1 receptor is hyperactive.

38. A pharmaceutical composition comprising a compound having a formula: 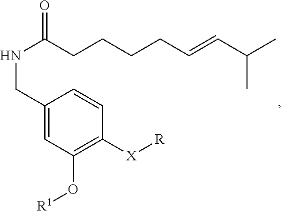
where
X is C(R¹)₂, O, N(R¹), or S;
R is —(CH₂)ₙYₐ(CH₂)ₘN(R¹)₃ or —C(O)(CH₂)ₙYₐ(CH₂)ₘN(R¹)₃;
R¹ is H or C₁₋₆ alkyl;
R² is H or C₁₋₆ alkyl;
R and R¹ are optionally substituted 1-6 times with halogen, ═O, —OR², —N(R²)₂, NO₂, C(O)R², C₂₋₆ alkenyl, and C₂₋₆ alkynyl;
n is an integer from 1 to 5;
m is an integer from 1 to 5;
a is 0 or 1; and
Y is O, N, or S
a stereoisomer, pharmaceutically acceptable salt, oxide, solvate, or ester thereof and a pharmaceutically acceptable carrier.
